(19) United States
(12) Patent Application Publication
(10) Pub. No.: US 2024/0307372 A1
(43) Pub. Date: Sep. 19, 2024

(54) COMPOSITIONS AND METHODS TO IMPROVE BENEFIT OF SUBOPTIMALLY ADMINISTERED THERAPEUCTICS FOR THE TREATMENT OF BENIGN AND NEOPLASTIC DISEASES, INFECTIONS, INFLAMMATORY, METABOLIC AND IMMUNOLOGIAL DISEASES

(71) Applicant: Escend Pharmaceuticals, Inc., Menlo Park, CA (US)

(72) Inventors: Saira S. Bates, Napa, CA (US); Dennis Brown, Palo Alto, CA (US); Shawnya Michaels, El Granada, CA (US)

(21) Appl. No.: 18/605,757

(22) Filed: Mar. 14, 2024

Related U.S. Application Data
(60) Provisional application No. 63/453,067, filed on Mar. 17, 2023.

(51) Int. Cl. A61K 31/4745; A61K 9/00; A61K 45/06; A61P 35/02

(52) U.S. Cl. CPC A61K 31/4745; A61K 9/0053; A61K 45/06; A61P 35/02

(57) ABSTRACT

Disclosed are novel compositions, methods to improve a suboptimal therapeutic agent's performance in patients with cancer, infections, inflammatory, metabolic, immunological diseases. Describe are improvements ranging from pharmaceutical ingredients, dosage forms, excipients, solvents, diluents, drug delivery systems, preservatives, more accurate drug administrations, dose determination/schedules, toxicity monitoring and amelioration, techniques or agents. Using suboptimal therapeutic(s)s in conjunction with radiation(s) and/or conventional chemotherapeutic agent(s)s or biotherapeutic agent(s) including antibodies, vaccines, cytokines, lymphokines, gene and antisense RNA therapies, etc., would provide unique approaches, significant treatment improvement circumventing or reducing toxicity, including techniques and tools identifying/predicting patients by phenotype or genotype determination(s). The disclosed invention also relates to using novel prodrugs, polymer conjugates, novel routes of administration, other agents to potentiate activity of the compound(s) or inhibit repair of suboptimal cellular effects or sublethal damage or to “push” the cell into more destructive cellular phases such as immune stimulation and apoptosis.

### CROSS REFERENCE TO RELATED APPLICATIONS

This application is a U.S. Utility Patent application which claims the benefit of priority to U.S. Provisional application No. 63/453,067, filed Mar. 17, 2023, entitled “Compositions and methods to improve the therapeutic benefit of suboptimally administered chemical compounds and biologic therapies including bisbenzylisoquinolines such as 6,6′,7,12-tetramethoxy-2,2′-dimethyl-berbaman and derivatives for the treatment of benign and neoplastic hyperproliferative disease conditions, infections, inflammatory, metabolic and immunological diseases” which is hereby incorporated by reference in its entirety.

### BACKGROUND OF THE DISCLOSED INVENTION

The search for and identification of cures for many life-threatening diseases that plague humans still remains an empirical and sometimes serendipitous process. While many advances have been made from basic scientific research to improvements in practical patient management, there still remains tremendous frustration in the rational and successful discovery of useful therapies particularly for life-threatening diseases such as cancer, immune-mediated diseases, inflammatory conditions, infection, etc.

Since the “War on Cancer” began in the early 1970's by the United States National Cancer Institute (NCI) of the National Institutes of Health (NIH), a wide variety of strategies and programs have been created and implemented to prevent, diagnose, treat and cure cancer and other life threatening disease conditions. One of the oldest and arguably most successful programs has been the synthesis and screening of small chemical entities (<1500 MW) for biological activity against cancer. These programs were organized to improve and streamline the progression of discovery and development events from chemical synthesis and molecular biology and biological screening to preclinical studies for the logical progression into human clinical trials with the hope of finding cures for the many types of life-threatening diseases including cancer. The synthesis and screening of hundreds of thousands chemical compounds from academic and industrial sources, in addition to the screening of natural products and extracts from prokaryotes, invertebrate animals, plants collections, etc. from all over the world as well as novel products exploited by molecular and synthetic biology methodologies has been and continues to be a major approach for the identification of novel lead structures as potential new and useful medicines. This is in addition to other programs including bio-therapeutics designed to stimulate the human immune system with adoptive immune cell transfers (e.g., CAR-T), vaccines, therapeutic antibodies, drug-antibody conjugates, cytokines, lymphokines, peptides, inhibitors of tumor blood vessel development (angiogenesis) or gene and antisense therapies to alter the genetic make-up of cancer cells or alter the immune system, etc.

The work supported by the NCI, other governmental agencies both domestic and foreign in academic or industrial research and development laboratories has resulted in an extraordinary body of biological, genomic, pharmacologic, biochemical, chemical and clinical information. In addition, large chemical and biological libraries have been created, as well as highly characterized in silico in vitro and in vivo biological screening systems that have been successfully used. However, from the tens of billions of dollars spent over the past fifty years supporting these programs both pre-clinically and clinically, only a limited number of therapeutics have been identified or discovered that have resulted in the successful development of useful pharmaceutical products. Nevertheless, the biological systems both in vitro and in vivo and the “decision trees” used to warrant further preclinical studies leading to phase I-III clinical trials have been validated. These drug screening programs, biological models, clinical trial protocols, etc., remain critical for the discovery and development of any new therapeutic agent.

Unfortunately, many of the compounds that have successfully met the preclinical testing and federal regulatory requirements for clinical evaluation were either unsuccessful or disappointing in human clinical trials. Many compounds were found to have untoward or idiosyncratic side-effects that were discovered during human clinical Phase I dose-escalation studies used to determine the maximum tolerated dose (MTD) and side-effect profile. In some cases, these toxicities or the magnitude of their toxicity were not identified or predicted in preclinical toxicology studies. In other cases, therapeutic agents where in vitro and in vivo studies suggested a potentially unique activity against a particular tumor type, molecular target or biological pathway were not successful in human Phase II clinical trials where specific examination of particular disease indications/types were evaluated in government sanctioned (e.g., U.S. FDA), IRB approved clinical trials. In addition, there are those cases where potential new agents were evaluated in randomized Phase III clinical trials where a significant clinical benefit could not be demonstrated have also been the cause of great frustration and disappointment. Finally, a number of compounds have reached regulatory approved commercialization but their ultimate clinical utility has been limited by poor efficacy as monotherapy (e.g., <25% response rates) and for untoward grade III and IV dose-limiting side-effects (e.g., myelosuppression, cardiotoxicity, gastrointestinal toxicities, cytokine storm effects) not clearly identified in regulatory clinical trials.

In many cases, after the great time and expense of developing and moving an investigational compound into human clinical trials and where clinical failure has occurred, the tendency has been to return to the laboratory to create a better analogs, look for agents with different structures but potentially related mechanisms of action, etc. In some cases, efforts have been made to try additional Phase I or II clinical trials in an attempt to make some improvement with the side-effect profile or therapeutic effect in selected patients or other disease indications. In many of those cases, the results did not realize a significant enough improvement to warrant further clinical development toward product registration. Even for commercialized products, their ultimate use can still be limited by suboptimal performance.

For example in oncology, with so few therapeutics approved for cancer patients and the realization that cancer is a collection of diseases with a multitude of etiologies, biological phenotypes or genotype with high rise for drug resistance and susceptible genomic mutations and that a patient's response and survival from therapeutic intervention is complex with many factors playing a role in the success or failure of treatment including disease indication, pathology stage related to invasion and metastatic spread, patient gender, age, health conditions, previous therapies or other illnesses, etc., the opportunity for significant cures rates without treatment morbidity in the near term remains elusive. Moreover, the incidence of cancer continues to rise such that over 1.6 million new cancer cases are estimated for 2015 in the United States by the American Cancer Society. In addition, with advances in diagnosis such as BRCA genetic testing and mammography for breast cancer and PSA tests for prostate cancer, more patients are being diagnosed at a younger age. For difficult to treat cancers, a patient's treatment options are often exhausted quickly resulting in a desperate need for additional treatment regimens. Even for the most limited of patient populations, any additional treatment opportunities would be of considerable value. This invention focuses on inventive compositions and methods for improving the therapeutic benefit of suboptimally administered therapeutic agents including bisbenzylisoquinolines such as 6,6′,7,12-tetramethoxy-2,2′-dimethyl-berbaman (tetrandrine) and derivatives.

### SUMMARY OF THE DISCLOSED INVENTION

This disclosed invention relates to novel compositions and methods to improve the utility of therapeutic agents with suboptimal performance in patients suffering with cancer, infections and immunological diseases. The disclosed invention describes novel improvements, pharmaceutical ingredients, dosage forms, excipients, solvents, diluents, drug delivery systems, preservatives, more accurate drug administrations, improved dose determination and schedules, toxicity monitoring and amelioration, techniques or agents to circumvent or reduce toxicity, techniques and tools to identify/predict those patients who might have a better outcome with a therapeutic agent by the use of phenotype or genotype determination through the use of diagnostic kits or pharmacokinetic or metabolism monitoring approaches. The disclosed invention also relates to the use of drug delivery systems, novel prodrugs, polymer conjugates, novel routes of administration, other agents to potentiate the activity of the compounds or inhibit the repair of suboptimal cellular effects or sublethal damage or to “push” the cell into more destructive cellular phases such as immune stimulation and apoptosis. In some case, the use of these suboptimal therapeutics in conjunction with radiations or other conventional chemotherapeutic agents or biotherapeutic agents such as antibodies, vaccines, cytokines, lymphokines, gene and antisense RNA therapies, etc., would provide novel approaches and potential significant treatment improvement.

In the inventive compositions and methods, the term suboptimal therapy includes agents where Phase I toxicity precluded further human clinical evaluation. It also includes those agents from Phase II trials where limited (e.g., <25% response rates) or no significant treatment responses were identified. Also, suboptimal therapy includes those agents, the subject of Phase III clinical trials the outcome of which was either medically or statistically not significant to warrant regulatory submission or approval by government agencies for commercialization for commercialized agents whose clinical performance (i.e. response rates) as a monotherapy are less than 25%, or whose side-effects are severe enough to limit wide utility. Agents with suboptimal clinical activity include but are not limited to the following: Small chemical therapeutics, natural products, biologics such as peptide, protein antibody drug conjugates, vaccines, including cell based therapies. More specifically, the inventive methods and compositions also focus on improvements for bisbenzylisoquinolines such as 6,6′,7,12-tetramethoxy-2,2′-dimethyl-berbaman (tetrandrine) and derivatives.

Tetrandrine (6,6′,7,12-tetramethoxy-2,2′-dimethylberbam) or TET is a bis-benzylisoquinoline alkaloid (chemical structure in FIG. 1), extracted from roots of the plant Stephania tetrandra S. Moore. ES-3000 (Tetrandrine) is being developed by Escend Pharmaceuticals, Inc. (Escend) for the treatment of relapsed or refractory AML and MDS. ASTX727 is an oral fixed dose combination of Decitabine (DEC) and Cedazuridine, recently FDA approved, for use in Myelodysplasia. The combination of two oral agents, with an established safety profile and potential combinatorial efficacy, would be a novel and much needed approach to treatment of intermediate and high risk MDS.

ASTX727 is the tablet referred to and is the investigational drug name for Inqovi [Decemberitabine (35 mg) and cedazuridine (100 mg)] tablet. The combination of ES-3000 at varying dosages can be administered with ASTX727. The ES-3000 schedule for administration can be at one of once, twice or three times/day and can vary from about 1-14 days to about 1-7 days in combination with administration of the standard does for ASTX727 (Inqovi) for about 1-5 days, about 1-4 days, about 1-3 days and about 1-2 days. The dosage of ES-3000 can be between 20-100 mg, 25-90 mg, 30-80 mg, 40-60 mg, as well as doses from about 20 mg, 30 mg, 40 mg, 50 mg, 60 mg, 70 mg, 80 mg, 90 mg, and 100 mg as well as 105 mg, 110 mg, 115 mg, 120 mg at once, twice or three times/day.

Anticancer activity has been demonstrated in vitro with TET against human breast, colon carcinoma, and hepatoma cell lines with an IC₅₀ range from 1.53-10.41 μM and inhibit drug efflux and increase intracellular drug accumulation in MDR cells. TET has also been shown to inhibit wnt/β-catenin signaling and tumor growth in human colorectal (HCT116) cells. In the same study, TET exhibited synergistic anticancer activity with 5-FU and reduced migration and invasion capabilities of HCT116 cells. [He 2011, Ng 2013]. In the NCI 60 cell line screen, a 50% growth inhibition (Log GI₅₀) ranged from −5.0 to −6.6 (0.25-10 μM was demonstrated in leukemia cell lines (Developmental Therapeutics Program). TET has been reported to have potent and specific activity in the reversal of P-glycoprotein mediated drug resistance. In a colorectal (CT-26) mouse xenografts, TET reduced tumor growth by inducing apoptosis [Wu 2010].

### BRIEF DESCRIPTION OF THE DRAWINGS

FIG. 1 illustrates the chemical structure of ES-3000, also known as Tetrandrine (TET).

FIG. 2 graphically illustrates the effect of TET treatment on K562 cells as percent cell viability.

FIG. 3 illustrates a PAGE showing ß-catenin protein exists as a doublet in all K562 cell extracts, including untreated cells.

FIG. 4 illustrates a PAGE showing a clear beta-Actin band evident in all extracts of K562 cells treated with 0 to 40 uM tetrandrine.

FIG. 5 presents a plot depicting beta-catenin level was significantly less in extracts of K562 cells treated with >10 uM tetrandrine.

FIG. 6 presents a plot depicting beta-Actin level in extracts of K562 cells treated with up to 40 uM tetrandrine was not affected significantly by drug treatment

FIG. 7A illustrates a bar graph of samples of circulating leukemic blood cells from recipient mice following treatment with Tetrandrine or Imatinib+Tetrandrine.

FIG. 7B illustrates a bar graph of samples of leukemic stem cells from recipient mice after treatment with Tetrandrine or Imatinib+Tetrandrine on day 8 after bone marrow transplantation.

FIG. 8 graphically illustrates electrophysiology measurements of CHO cells stably transfected with hERG to examine the potential of a test article to inhibit hERG. Post-compound currents expressed as a percentage of pre-compound currents plotted against concentration for each compound.

### DETAILED DESCRIPTION

Tetrandrine (NSC 77037, d-tetrandrine, TET) or ES-3000 is a bis-benzylisoquinoline alkaloid, extracted from roots of the plant Stephania tetrandra S. Moore. ES-3000 (TET) is being developed by Escend Pharmaceuticals, Inc. for the treatment of relapsed or refractory (R/R) acute myeloid leukemia (AML).

The are several known mechanism of action of TET including inhibition of calcium channels and activated big potassium channels [Wang 1995], which are commonly overexpressed in malignancies and have been demonstrated to play a role in cancer and leukemia [Arcangeli 2010, Arcangeli 2012, Huber 2013, Lang 2014]. TET has also been reported to reversal of P-glycoprotein (P-gp) mediated drug resistance [Zhu 2005] and though, TET itself is not a substrate of Pgp, it directly inhibits the function of Pgp in a dose-dependent manner, without altering the protein expression level of Pgp [Susa 2012]. More recently, TET has been demonstrated to inhibit the Wnt/β-catenin pathway [He 2011, Xu 2012], which has been identified as a new target for AML. Wnt/β-catenin is essential for the survival and self-renewal of leukemic stem cells in acute myeloid leukemia (AML) [Simon 2005, Griffiths 2010, Wang 2010]. In addition, TET has been demonstrated to bind competitively to calmodulin (CaM) and may be a novel CaM antagonist [Ma 2013]. Calmodulin protein dependent kinase (CaMKIIγ) is overexpressed in leukemic stem cells and blast cells, and regulates the Wnt/β-catenin and STAT3 pathways [Si 2008].

Published clinical data support the safety and efficacy of TET in oncology patients provides another compelling rationale for clinically assessing TET in leukemia. A phase I dose escalation study was conducted by the National Cancer Institute (NCI) under IND 9613 (also known at 089613). Thirty-two patients with advanced solid tumors were administered IV doses of TET ranging from 50 to 875 mg/m2. The recommended dose for a Phase II study was 300 mg/m2 IV, single dose repeated weekly; or 200 mg/m2 IV, daily ×5, repeated every 2 weeks (IND 9613 Tetrandrine Investigators Reports). Further supporting published reports include clinical studies using TET under the trademarked name CBT-1® in the US] Oldham 1998, Oldham 2000, Kelly 2012] and two studies in China [Xu 2006, Liu 2012]. The drug known as CBT-1 is the same drug substance (NSC 77037) used in US Phase I study (IND #089613). CBT-1 had been publicly disclosed as NSC-77037 (tetrandrine) by [Fanelli 2016].

Drug substance; Tetrandrine and drug product, Tetrandrine tablet (20 mg) has been manufactured under GMP compliance by Jinhua Conba since 1996, which is the largest manufacture of tetrandrine in China.

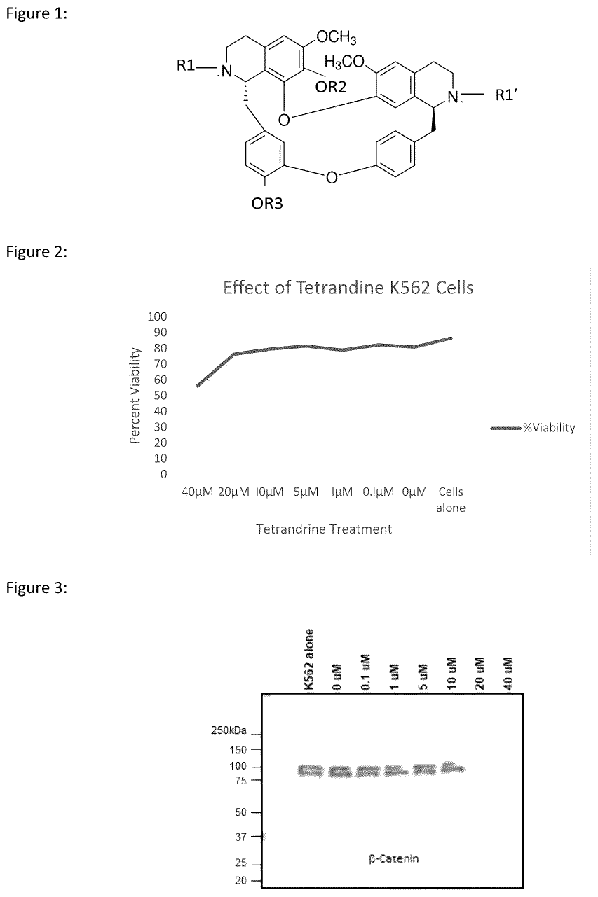

Chemical Structure of Tetrandrine is illustrated in FIG. 1. Tetrandrine is the generic name for NSC 77037, d-tetrandrine with the chemical structure written as,6′,7,12-tetramethoxy-2,2′-dimethyl-berbaman. The IUPAC name is 9,20,21,25-tetramethoxy-15,30-dimethyl-7,23-dioxa-15,30-diazaheptacyclo[22.6.2.2³,⁶0.1⁸, ¹²0.1¹⁴, ¹⁸0.0²⁷,³¹0.0²²,³³]hexatriaconta-3,5,8(34),9,11,18,20,22(33),24(32),25,27(31),35-dodecaene, C₃₈H₄₂N₂O₆ is the chemical formula with a molecular weight of 622.76 and has CAS Registry No. 518-34-3.

This disclosed invention relates to novel compositions and methods to improve the utility of chemical agents, biological therapies including bisbenzylisoquinolines such as 6,6′,7,12-tetramethoxy-2,2′-dimethyl-berbaman (tetrandrine) and derivatives with suboptimal performance for patients with cancer, infection and immunological diseases. The disclosed invention describes the novel development of improved pharmaceutical ingredients, dosage forms, excipients, solvents, diluents, drug delivery systems, preservatives, more accurate drug administrations, improved dose determination and schedules, toxicity monitoring and amelioration, techniques or agents to circumvent or reduce toxicity, techniques and tools to identify/predict those patients who might have a better outcome with a therapeutic agent by the use of phenotype or genotype determination through the use of diagnostic kits or pharmacokinetic or metabolism monitoring approaches , the use of drug delivery systems, novel prodrugs, polymer conjugates, novel routes of administration, other agents to potentiate the activity of the compounds or inhibit the repair of suboptimal cellular effects or sub-lethal damage or to “push” the cell into more destructive cellular phases such as apoptosis. In some cases, the inventive examples include the use of these sub-optimal therapeutics in conjunction with radiations or other conventional chemotherapeutic agents or biotherapeutic agents such as antibodies, vaccines, cytokines, lymphokines, gene and antisense therapies, etc.

As used herein, “suboptimal” refers to “less that desired” and the terms “suboptimal therapy” and “suboptimal therapeutics” include agents where Phase I toxicity precluded further human clinical evaluation, agents from Phase II trials where limited or no significant tumor responses were identified, and agents form Phase III clinical trials, whose outcome was either medically or statistically not significant to warrant submission or approval by regulatory agencies for commercialization or commercialized agents whose response rates as a monotherapy are less than 25% or whose side-effects are severe enough to limit wider utility. In addition, it includes, allergic reaction, adverse or fatal side effects, organ damage, due to suboptimal therapeutics. Agents with suboptimal activity include but are not limited to the following: bisbenzylisoquinolines including 6,6′,7,12-tetramethoxy-2,2′-dimethyl-berbaman (tetrandrine) and derivatives, Avastin® (bevacizumab), Rituxan® (rituximab), Neavar® (sorafenib), dasatinib, imatinib, nilotinib, Provenge® (sipuleucel-T), Tarceva® (erlotinib), and Iressa® (gefitinib). More specifically, the inventive methods and compositions also focus on improvements for bisbenzylisoquinolines including 6,6′,7, 12-tetramethoxy-2,2′-dimethyl-berbaman (tetrandrine) and derivatives.

The following disclosed invention is further illustrated by the following examples, which should not be construed as further limiting. The contents of all references, provided manuscripts, pending patent applications and published patents, cited throughout this application are hereby expressly incorporated by reference.

### EXAMPLES

Suboptimal therapeutics: Examples of compounds with suboptimal therapeutic activity may include antimetabolites, DNA/nucleic acid binding/reactive agents, topoisomerase inhibitors, anti-tubulin agents, signal transduction inhibitors, protein synthesis inhibitors, inhibitors of DNA transcribing enzymes, DNA/RNA intercalating agents, DNA minor groove binders, drugs that block steroid hormone action, photochemically active agents, immune modifying agents, hypoxia selective cytotoxins, chemical radiation sensitizers and protectors, antisense nucleic acids, oligonucleotides and polynucleotides therapeutic agents, immune modifying agents, antitumor antibiotics, biotherapeutics, biologic agents such as cancer vaccines, antibody therapies, cytokines, lyphokines, gene therapies, nucleic acid therapies, cellular therapies, etc.

Specific examples include but are not limited to: fluoropyrimidines, thiopurines, inhibitors of nucleoside diphosphate reductase, 2′-deoxyribonucleoside analogs, nucleosides, folic acid analogs, methotrexate, 6-diazo-5-oxo-norleucine, L-asparaginase, N-(phosphoacetyl)-L-aspartic acid, nitrogen mustard, mechlorethamine, chlorambucil, melphalan, cyclophosphamide, estramustine, platinum complexes, nitrosoureas, BCNU, BCNU wafer (Gliadel®), CCNU, streptozotocin, alkyl sulfonates, busulfan, clomesone, triazenylimidazoles and related triazenes, mitozolomide, temozolomide (Temodar), aziridines, tris(1-aziridinyl)phosphine sulfide, aziridinylphosphines, 3,6,-diaziridinyl-2,5-bis(carboethoxyamino)-1,4-benzoquinone (Diaziquone®)(AZQ), AZQ analogs, bendamustine (Treanada®) procarbazine, hexamethylamine, topoisomerase I inhibitors, camptothecin, camptothecin analogs, topoisomerase II inhibitors, anthracyclines, doxorubicin, epirubicin, etoposide, DNA intercalating agents, amsacrine, CI-921, 1′-carbamate analogs of amsacrine, 9-aminoacridine-4-carboxamides, acridine carboxamide, tricyclic carboxamides, 1-nitroacridine, acridine derivatives, diacridines, triacridines, podophyllotoxins, ellipticine, merbarone, benzisoquinolinediones, etoposide, teniposide, aminoanthraquinones, inhibitors of DNA-transcribing enzymes, transcription inhibitors, replication inhibitors, RNA replication inhibitors, polymerase inhibitors, rifamycins, actinomycins, DNA minor groove binding compounds, Hoechst 33258, mitomycins, CC-1065, mithramycins, chloromycins, olivomycins, phthalanilides, anthramycins, antimitotic agents, vinca alkaloids, vinblastine and analogs, vincristine and analogs, navelbine, colchicine and analogs, bleomycin and analogs, estramustine, aromatase inhibitors, tamoxifen, LHRH antagonists and analogs, porfimer, hematoporphyrins, electron-affinic oxygen mimetics, nitoaromatics, nitroheterocyclics, nitroimidizaoles, tirapazamine, mitomycins, menadione and analogs, napthoquinones, aziridoquinones, amine oxides, N-oxides, metal complexes, bioreductive agents, bioreductive amino acid substituted alkylating agents, metal complexes, radiation sensitizers, radiation protectors, antisense agents, antigene agents, transcription factor inhibitors, ODN complexes, ribozymes, double stranded RNA, antitumor antibiotics, acivicin, aclararubicin, acodazole, acronycine, adozelesin, alanosine, allopurinol, altretamine, aminoglutethimide, amonafide, amsacrine, androgens, anguidine, aphidicolin glycinate, 5-azacitidine, azathioprine, Baker's Antifol, beta-2′-deoxythioguanosine, bisantrene HCl, bleomycin sulfate, busulfan, buthionine sulfoximine (BSO), BWA 773U82, BW 502U83 HCl, BW 7U85 mesylate, caracemide, carbetimer, carboplatin, carmustine, chloambucil, chloroquinoxaline sulfonamide, chlorozotocin, chromomycin A3, cisplatin, cladribine, carboplatin, oxaliplatin, rhodamine compounds, corticosteroids, CPT-11, cristanol cyclocytidine, cyclophosphamide, cytarabine, cytembena, dabis maleate, dacarbazine, dactinomycin, daunorubicin HCl, deazauridine, dexrazoxane, (DAG), diaziquone (AZQ), didemnin B, diethyldithiocarbamate, diglycoaldehyde, dihydro-5-azacytidine, doxorubicin, echinomycin, edatrexate, edelfosine, eflornithine, elsamitrucin, epirubicin, esorubicin, estramustine phosphate, estrogens, etanidazole, ethiofos, etoposide, fadrazole, fazarabine, fenretinide, finasteride, flavone acetic acid, floxuridine, fludarabine phosphate, 5-fluorouracil, flutamide, gallium nitrate, gemcitabine, goserelin acetate, hepsulfam, hexamethylene bisacetamide, hydrazine sulfate, 4-hydroxyandrostenedione, hydroxyurea, idarubicin HCl, ifosfamide, 4-ipomeanol, iproplatin, isotretinoin, leuproloide acetate, levamisole, liposomal daunorubicin, liposomal doxorubicin, lomustine, lonidamine, maytansine, mechloethamine hydrochloride, melphalan, menogaril, 6-mercaptopurine, mesna, methotrexate, N-methylformamide, mifepristone, acetylsaracolysin L-Leucine, mitomycin C, mitotane, mitoxantrone hydrochloride, nabilone, nafoxidine, neocarzinostatin, octreotide acetate, ormaplatin, oxaliplatin, paclitaxel, pala, pentostatin, piperazinedione, pipobroman, pirarubicin, piritrexim, piroxantrone hydrochloride, plicamycin, porfimer sodium, predimustine, procarbazine, progestins, pyrazofurin, razoxane, sargramostim, semustine, spirogermanium, streptonigrin, streptozocin, sulofenur, suramin sodium, tamoxifen, taxotere, tegafur, teniposide, terephthalamidine, teroxirone, thioguanine, thiotepa, thymidine, tiazofurin, topotecan, tormifene, treinoin, trifluoroperazine hydrochloride, trifluridine, trimetrexate, uracil mustard, vinblastine sulfate, vincristine sulfate, vindesine, vinorelbine, vinzolidine, Yoshi 864, zorubicin 2-Cl-2′ deoxyadenosine, 3-deazauridine, 4-nitroestrone, 6-methylmercaptopurine riboside, 9-aminocamotothecin ,nitrocamptothecin , irinotecan, CPT-11, acivicin, acodazole HCl, ADR-529, ICRF-187, amasacrine, aminothiadiazole, ADTA, amonafide, antibiotic FR901228, aphidicolin glycinate, azacitidine, AZT, bizelesin, brefeldins, wortmannins, canthardins, bromodeoxyuridines, bryostatin, BSO, CAI, caracemide, carboplatin, chlorosulfaquinoxaline, sulfonamide, cisplatin, clomesone, cyclocytidine HCl, cyclodisone, cyclopentenylcytosine, deoxyspergualin, DHAC, diaziquone, didemnin B dideoxy-beta-fluorouracil, dideoxyadenosine, dideoxyinosine, dihydrotriazine benzene sulfonyl fluoride, dolastatin 10, ecteinascidin 743, ctanidazole, ethiofos (WR-2721), fazarabine, flavone acetic acid, flavopiridol, fludarabine phosphate, fostriecin, gallium nitrate, genistein, hepsulfam, HMBA, hydrazine sulfate, iododeoxyuridine, ipomeanol, KNI-272, leucovorin calcium, levamisole, melphalan, menogaril, merbarone, methotrexate, misonidazole, mitoxantrone HCl, mitozolomide, N-methylformamide, 06-benzylguanine, PALA, pancratistatin, penclomedine, pentamethylmelamine HCl, pentamidine isethionate, pentostatin, perillyl alcohol, phyllanthoside, pibenzimole HCl, piroxantrone, pyrazine diazohydroxide, pyrazoloacridine, quinocarmycins, rebeccamycins, rhizoxin, semustine (methyl CCNU), suramin sodium, Nexavar®, imatinib, dasatinib (Sprycell®), bosutinib, nilotinib, decitabine, 5-azacitidine (Vidaza®) Homoharringtonine (HHT, omacetaxine), Taxol®, taxotere, temozolomide (Temodar®), terephthalamidine, teroxirone, thioguanine, thymidine, tiazofurin, TMCA, topotecan, 5-fluorouracil, methotrexate, cyclophosphamide, ras inhibitors, farnesylation inhibitors, bromodeoxyuridine, tetracycline compounds, arsenic trioxide, combretastatins, 2-methoxyestradiol, thalidomide and analogs, cephalotaxine derivatives, stributyrin, triciribine phosphate, trimetrexate, UCN-01, 7-hydroxystaurosporine, uridine, lycurium, ritrosulfan, artemisinin, artesunate, lonidamine, mesna, bromomannitol, hydrazine sulfate, pipobroman, phenesterin, pyrazine diazohydroxide, cytembena, spirogermanium, terephthalamidine, bufalin, gemcitabine, FMDC, colchicine, thiocolchicine, colchicine analogs, LHRH analogs, paclitaxel, MGBG, antibody therapies such as Avastin®, Herceptin®, Rituxan®, Erbitux®. Also included are such as bisbenzylisoquinolines such as 6,6′,7,12-tetramethoxy-2,2′-dimethyl-berbaman (tetrandrine) and derivatives, (DAG, dianhydrodulcitol) (DBD), yervoy, keytrudu, PD-1 (effectors/inhibitors/antibodies), PD-L1 (effectors/inhibitors/antibodies), CTLA-4 antibodies, Provenge, CAR-T therapeutics, PARP inhibitors, BTK inhibitors, BRAF inhibitors, antibody drug conjugates, Interleukins, Interferons, CD-20 antibodies, PTEN inhibitors, checkpoint inhibitors, indoleamine 2,3-dioxygenase (IDO), hypomethylating agents, 5 azacytidine, decitiabine , decitiabine combined with cedazuridine (Inqovi) antiarthritic agents, anti-TNF antibodies, CAR-T cells, tumor vaccines.

Dose modification: Improvements for suboptimal therapeutics including bisbenzylisoquinolines such as 6,6′,7, 12-tetramethoxy-2,2′-dimethyl-berbaman (tetrandrine) and derivatives can be made by by one or more of the following alone or in combination: alterations to the time that the compound is administered, the use of dose-modifying agents that control the rate of metabolism of the compound, normal tissue protective agents, etc.
a) Examples include but are not limited to by one or more of the following alone or in combination: variations of infusion schedules (e.g., bolus i.v. versus continuous infusion), the use of lymphokines (e.g., G-CSF, GM-CSF, EPO) to increase leukocyte count for or prevent anemia caused by myelosuppressive agents, rescue agents such as leucovorin for 5-FU or thiosulfate for cisplatin treatment.
b) Specific inventive examples for bisbenzylisoquinolines such as 6,6′,7,12-tetramethoxy-2,2′-dimethyl-berbaman (tetrandrine) and derivatives include but are not limited to by one or more of the following alone or in combination: i.v. infusion for hours to days; biweekly, tri-weekly, monthly administration; doses greater than 4 mg/kg/day; progressive escalation of dosing from 1 mg/kg/day to greater than 4 mg/kg/day based on patient tolerance; doses less than 1 mg/kg/day for greater than 14 days; use of polyamine to modulate metabolism: use of eflornithine to modulate metabolism; selected and intermittent boost dose administrations; bolus single and multiple doses escalating from 1 mg/kg; oral doses below 1 or above 1.9 mg/kg/day.

Route of administration: Improvements for suboptimal therapeutics including but are not limited to bisbenzylisoquinolines such as 6,6′,7, 12-tetramethoxy-2,2′-dimethyl-berbaman (tetrandrine) and derivatives are made by alterations in the route that the compound is administered including one or more of the following alone or in combination: changing route from oral to intravenous administration and vice versa. The use of specialized routes such as subcutaneous, intramuscular, intraarterial, intraperitoneal, intralesional, intralymphatic, intratumoral, intrathecal, intravesicular, intracranial. Specific inventive examples for bisbenzylisoquinolines such as 6,6′,7,12-tetramethoxy-2,2′-dimethyl-berbaman (tetrandrine) and derivatives include: topical; intravesicular for bladder cancer; oral administration; slow release oral delivery; slow release oral delivery; extended release oral delivery; rectal delivery; intrathecal; intraarterial; continuous infusion; intermittent infusion.

Schedule of administration: Improvements for suboptimal therapeutics including but are not limited to bisbenzylisoquinolines such as 6,6′,7,12-tetramethoxy-2,2′-dimethyl-berbaman (tetrandrine) and derivatives are made by alterations to the time that the compound is administered. General examples include: changing from a monthly administration to a weekly or daily dosing or variations of the schedule. Specific inventive examples for bisbenzylisoquinolines such as 6,6′,7,12-tetramethoxy-2,2′-dimethyl-berbaman (tetrandrine) and derivatives include: daily; weekly administration; weekly for three weeks, weekly for two weeks, biweekly; biweekly for three weeks with a 1 to 2-week rest period; intermittent boost dose administration; daily for one week then once per week for multiple weeks.

Indications for use: Improvements for suboptimal therapeutics including bisbenzylisoquinolines such as 6,6′,7,12-tetramethoxy-2,2′-dimethyl-berbaman (tetrandrine) and derivatives are made by alterations in the types of disease, clinical stage of disease that the compound is administered. General examples include: the use of solid tumor agents for leukemias and vice versa, the use of antitumor agents for the treatment of benign hyperproliferative disease such as psoriasis or benign prostate hypertrophy. Specific inventive examples for bisbenzylisoquinolines such as 6,6′,7,12-tetramethoxy-2,2′-dimethyl-berbaman (tetrandrine) and derivatives include but are not limited to: leukemias (acute and chronic, AML, ALL, CLL, CML); myelodysplastic syndrome (MDS); leukemia with MLL translocations (11q23); leukemia enriched in FLT3 or NPM somatic mutations; leukemia with genomic alterations in IDHI or DNMT3A genes; angiogenic diseases; benign prostate hypertrophy; psoriasis; gout; autoimmune conditions; prevention of transplantation rejection; restenosis prevention in cardiovascular disease; mycoses fungoides; use in bone marrow transplantation; as an anti-infective; treatment for AIDS; treatment for lymphoma; mantle cell lymphoma; meningeal leukemia; malignant meningitis; cutaneous T cell lymphoma; Barret's esophagus; anaplastic gliomas; triple negative breast cancer; Braf mutated melanoma; BTK resistant CLL; lymphoma; Chordoma; KRAS mutated colon cancer; RAS mutated lung cancer; pancreatic cancer, rheumatoid arthritis, osteo arthritis, Covid infection.

Disease stages: Improvements for suboptimal therapeutics including bisbenzylisoquinolines such as 6,6′,7,12-tetramethoxy-2,2′-dimethyl-berbaman (tetrandrine) and derivatives are made by alterations in the stage of disease at diagnosis/progression that the compound is administered. General examples include: the use of chemotherapy for non-resectable local disease, prophylactic use to prevent metastatic spread or inhibit disease progression or conversion to more malignant stages. Specific inventive examples for bisbenzylisoquinolines such as 6,6′,7,12-tetramethoxy-2,2′-dimethyl-berbaman (tetrandrine) and derivatives include: chronic and acute myeloid leukemias, pre-leukemic conditions such as myelodysplastic syndrome, localized polyp stage colon cancer; leukoplakia in the oral cavity; angiogenesis inhibition to prevent or limit metastatic spread; chronic myeloid leukemia with molecular responses less than MR4.5; myeloid leukemias with measurable minimal residual disease.

Other indications: Improvements for suboptimal therapeutics including bisbenzylisoquinolines such as 6,6′,7,12-tetramethoxy-2,2′-dimethyl-berbaman (tetrandrine) and derivatives are made by using the compound for non-malignant diseases and conditions. General examples include: premalignant conditions, benign hyperproliferative conditions, treatment of infections, parasites, usage to relieve pain, control of pleural effusions. Specific inventive examples for bisbenzylisoquinolines such as 6,6′,7,12-tetramethoxy-2,2′-dimethyl-berbaman (tetrandrine) and derivatives include: use as antiinfectives, antivirals; antibacterials; for pleural effusions; antifungals; antiparasitics; eczema; shingles; condyloma; anti HPV; anti-HSV, early and late stage MDS (Myelodysplastic syndrome), polycythemia vera, autoimmune disease, arthrititis, Covid infections

Patient selection: Improvements for suboptimal therapeutics including bisbenzylisoquinolines such as 6,6′,7,12-tetramethoxy-2,2′-dimethyl-berbaman (tetrandrine) and derivatives are made by alterations to the type of patient that would best tolerate or benefit from the use of the compound. General examples include: use of pediatric doses for elderly patients, altered doses for obese patients; exploitation of co-morbid disease conditions such as diabetes, cirrhosis, etc. that may uniquely exploit a feature of the compound. Specific inventive examples for bisbenzylisoquinolines such as 6,6′,7,12-tetramethoxy-2,2′-dimethyl-berbaman (tetrandrine) and derivatives include: patients with minimal residual disease or resistant disease; patients with cancer stem cells; patients with disease conditions with high levels of metabolic enzymes, histone deacetylase, protein kinases, ornithine decarboxylase; patients with disease conditions with low levels of metabolic enzymes, histone deacetylase, protein kinases, ornithine decarboxylase; patients with low or high susceptibility to thrombocytopenia, neutropenia; patients intolerant of GI toxicities; over or under expression of jun, GPCR's and signal transduction proteins, VEGF, prostate specific genes, protein kinases, telomerase; patients with high expression of beta-catenin in cancerous cells; patients with high expression of activated calmodulin-dependent protein kinase II-gamma (CaMKIIγ) in cancerous cells.

Patient/disease phenotype: Improvements for suboptimal therapeutics including bisbenzylisoquinolines such as 6,6′,7,12-tetramethoxy-2,2′-dimethyl-berbaman (tetrandrine) and derivatives are made by more precise identification of a patient's ability to tolerate, metabolize and exploit the use of the compound. General examples include: use of diagnostic tools and kits to better characterize a patients ability to process/metabolize a chemotherapeutic agent or their susceptibility to toxicity caused by potential specialized cellular, metabolic, organ system phenotypes: Specific inventive examples for bisbenzylisoquinolines such as 6,6′,7,12-tetramethoxy-2,2′-dimethyl-berbaman (tetrandrine) and derivatives include: diagnostic tools, techniques, kits and assays to confirm a patient's particular phenotype and for the measurement of minimal residual disease, resistant disease or cancer stem cells, metabolism enzymes and metabolites, histone deacetylase, protein kinases, ornithine decarboxylase, VEGF, prostate specific genes, telomerase, jun GPCR's; surrogate compound dosing or low dose drug pre-testing for enzymatic status; beta-catenin; calmodulin dependent protein kinase II-gamma (CaMKII-gamma); phospho-CaMKII-gamma.

Patient/disease genotype: Improvements for suboptimal therapeutics including bisbenzylisoquinolines such as 6,6′,7,12-tetramethoxy-2,2′-dimethyl-berbaman (tetrandrine) and derivatives are made by testing and analyzing a patient's genotype for unique features that may be of value to predict efficacy, toxicity, metabolism, etc. General examples include: biopsy samples of tumors or normal tissues (e.g., white blood cells) may also be taken and analyzed to specifically tailor or monitor the use of a particular drug against a gene target, unique tumor gene expression pattern, SNP's (single nucleotide polymorphisms), to enhance efficacy or to avoid particular drug-sensitive normal tissue toxicities. Specific inventive examples for bisbenzylisoquinolines such as 6,6′,7,12-tetramethoxy-2,2′-dimethyl-berbaman (tetrandrine) and derivatives include: diagnostic tools, techniques, kits and assays to confirm a patient's particular genotype; gene/protein expression chips and analysis; Single Nucleotide Polymorphisms (SNP's) assessment; SNP's for histone deacetylase; ornithine decarboxylase; S-adenosyl methionine GPCR's; protein kinases; telomerase; VEGF; prostate specific genes; jun; identification and measurement of metabolism enzymes and metabolites; mutation in wild type and mutated genes.
a) For myelodysplastic syndrome (MDS) and acute myeloid leukemia (AML) mutational targets involved with DNA methylations, chromatin modifications, RNA splicing, transcription, signal transduction, cohesion regulation, DNA repair, cytogenetic abnormalities, TP53. MRAS, RUNX1, ATRX KRAS, KIT mutations, etc.
b) Impacts on the epitranscriptome modifications can cause changes in the fate or function of RNA.
c) Impacts on leukemic stems cells (LSC) and their microenvironment. Targeting LSC's with enriched expression of the CD34 pos and CD38 neg fraction. Those cells with a higher expression of CD25,32,44,96,123, GPR56, PU.1 gene. 1 Rap, N-cadherin and Tie2.
d) Impacts on chemotherapeutic resistance mechanisms include MDR (multidrug resistance efflux pumps, pGP (p-glyco protein) adenosine triphosphate-binding cassette transporter superfamily.
e) Impacts on the inflammasome including sensors AIM2, NLRP3, adaptor caspase 1, IFN-1 alpha, beta levels and modulation, IL-1beta, SOCS1 upregulation, NFkB.
f) Impacts on minimal residual disease (MRD) defined as 1 MRD cell per 10,000 cells out of all bone marrow mono-nuclear cells.
g) Impacts on fibrosisincluding alteration of fibroblasts, and myofibroblasts, synthesi and degradation of extracellular matrix (ECM) components.

Pre/post treatment preparation: Improvements for suboptimal therapeutics including bisbenzylisoquinolines such as 6,6′,7,12-tetramethoxy-2,2′-dimethyl-berbaman (tetrandrine) and derivatives are made by specialized preparation of a patient prior to or after the use of a therapeutic agent. General examples include: induction or inhibition of metabolizing enzymes, specific protection of sensitive normal tissues or organ systems. Specific inventive examples for bisbenzylisoquinolines such as 6,6′,7,12-tetramethoxy-2,2′-dimethyl-berbaman (tetrandrine) and derivatives include: tyrosine kinase inhibitors (TKIs); daunorubicin and cytarabine or liposomal formulations of daunorubicin and cytarabine; checkpoint inhibitors such as anti-PDI antibodies, anti-PD-LI antibodies; CAR-T cells; etoposide; colchicine or analogs; use of diuretics such as probenecid; uricase; non-oral use of nicotinamide; sustained release forms of nicotinamide; inhibitors of polyADP ribose polymerase; caffeine; leucovorin rescue; infection control; antihypertensives; alteration of stem cell populations; pretreatment to limit or prevent graft vs. host (GVH) cytokine storm reactions; post-treatment with chemotherapeutics; post-treatment with tyrosine kinase inhibitors; post-treatment with immunotherapeutics.

Toxicity management: Improvements for suboptimal therapeutics including bisbenzylisoquinolines such as 6,6′,7,12-tetramethoxy-2,2′-dimethyl-berbaman (tetrandrine) and derivatives are made by use of additional drugs or procedures to prevent or reduce potential side-effects or toxicities. General examples include: the use of anti-emetics, anti-nausea, hematological support agents to limit or prevent neutropenia, anemia, thrombocytopenia, vitamins, antidepressants, treatments for sexual dysfunction, etc. Specific inventive examples for bisbenzylisoquinolines such as 6,6′,7,12-tetramethoxy-2,2′-dimethyl-berbaman (tetrandrine) and derivatives include: the use of colchicine or analogs; use of diuretics such as probenecid; use of uricase; non-oral use of nicotinamide; sustained release forms of nicotinamide; use of inhibitors of polyADP-ribose polymerase; use of caffeine; leucovorin rescue; sustained release allopurinol; non-oral use of allopurinol; bone marrow transplant stimulants; blood; platelet infusions; Neupogen; G-CSF; GM-CSF; filgrastim; pain management; antiinflammatories; fluids; corticosteroids; insulin control medications; anti-pyretics; anti-nausea treatments; anti-diarrhea treatment; N-acetyl cysteine; antihistamines, limit or prevent mucositis; limit or prevent GVH reactions; limit or prevent cytokine storm reactions; anti-fungals; sodium thiosulfate; N-acetylcysteine; glutathione; platelet transfusions; hypotensives; hypertensives.

Pharmacokinetic/pharmacodynamic monitoring: Improvements for suboptimal therapeutics including bisbenzylisoquinolines such as 6,6′,7,12-tetramethoxy-2,2′-dimethyl-berbaman (tetrandrine) and derivatives are made by the use of monitoring drug levels after dosing in an effort to maximize a patient's drug plasma level, to monitor the generation of toxic metabolites, monitoring of ancillary medicines that could be beneficial or harmful in terms of drug-drug interactions. General examples include: the monitoring of drug plasma protein binding, etc. Specific inventive examples for bisbenzylisoquinolines such as 6,6′,7,12-tetramethoxy-2,2′-dimethyl-berbaman (tetrandrine) and derivatives include: multiple determinations of drug plasma levels; multiple determinations of metabolites in the blood or urine; beta-catenin levels; BK channel levels; gene sequencing; immune effectors; CamKII-gamma; phospho-CamKII-gamma.

Drug combinations: Improvements for suboptimal therapeutics including bisbenzylisoquinolines such as 6,6′,7,12-tetramethoxy-2,2′-dimethyl-berbaman (tetrandrine) and derivatives are made by exploiting unique drug combinations that may provide a more than additive or synergistic improvement in efficacy or side-effect management. These can be administered separately or in the same dosage form. General examples include: amino acid substituted alkylating agents with anti-metabolites, topoisomerase inhibitors with antitubulin agents. Specific inventive examples for bisbenzylisoquinolines such as 6,6′,7,12-tetramethoxy-2,2′-dimethyl-berbaman (tetrandrine) and derivatives include: signal transduction inhibitors; tyrosine kinase inhibitors such as imatinib, dasatinib, nilotinib and bosutinib; topoisomerase inhibitors; fraudulent nucleosides; fraudulent nucleotides; thymidylate synthase inhibitors; cisplatin or platinum analogs; amino acid substituted alkylating agents such as the nitrosoureas (BCNU, Gliadel wafers, CCNU), bendamustine (Treanda) Temozoloimide (Temodar); anti-tubulin agents; antimetabolites; berberine; apigenin; amonafide; colchicine and analogs; genistein; etoposide; cytarabine; campothecins; vinca alkaloids; topoisomerase inhibitors; 5-fluorouracil; gemcitabine; curcumin; NFKappaB inhibitors; rosmarinic acid; biological therapies such as antibodies such as Avastin, Rituxan, Herceptin, Erbitux; PD-1 and PD-L1 inhibitors; prendimustine; DNA and RNA therapeutics; Braf inhibitors; BTK inhibitors; 5-azacytidine; decitabine; indoleamine 2,3-dioxygenase; daunorubicin; doxycycline; cytarabine; liposomal formulations of daunorubicin and cytarabine; doxorubicin; checkpoint inhibitors; CAR-T cells; indoleamine 2,3-dioxygenase, hypomethylating agents

Chemosensitization: Improvements for suboptimal therapeutics including bisbenzylisoquinolines such as 6,6′,7,12-tetramethoxy-2,2′-dimethyl-berbaman (tetrandrine) and derivatives are made by exploiting them as chemosensitizers where no measureable activity is observed when used alone but in combination with other therapeutics a more than additive or synergistic improvement in efficacy is observed. General examples include: misonidazole with amino acid substituted alkylating agents, tirapazamine with cisplatin. Specific inventive examples for bisbenzylisoquinolines such as 6,6′,7,12-tetramethoxy-2,2′-dimethyl-berbaman (tetrandrine) and derivatives include: signal transduction inhibitors; tyrosine kinase inhibitors such as imatinib, dasatinib, nilotinib and bosutinib; topoisomerase inhibitors; fraudulent nucleosides; fraudulent nucleotides; thymidylate synthase inhibitors; cisplatin or platinum analogs; amino acid substituted alkylating agents such as the nitrosoureas (BCNU, Gliadel wafers, CCNU), bendamustine (Treanda) Temozoloimide (Temodar); anti-tubulin agents; antimetabolites; berberine; apigenin; amonafide; colchicine and analogs; genistein; ctoposide; cytarabine; campothecins; vinca alkaloids; topoisomerase inhibitors; 5-fluorouracil; gemcitabine; curcumin; NFKappaB inhibitors; rosmarinic acid; biological therapies such as antibodies such as Avastin, Rituxan, Herceptin, Erbitux; PD-1 and PD-L1 inhibitors; prendimustine; DNA and RNA therapeutics; Braf inhibitors; BTK inhibitors; 5-azacytidine; decitabine; indolcamine 2,3-dioxygenase; daunorubicin; doxycycline; cytarabine; liposomal formulations of daunorubicin and cytarabine; doxorubicin; checkpoint inhibitors; CAR-T cells; indoleamine 2,3-dioxygenase.

Chemopotentiation: Improvements for suboptimal therapeutics including bisbenzylisoquinolines such as 6,6′,7,12-tetramethoxy-2,2′-dimethyl-berbaman (tetrandrine) and derivatives are made by exploiting them as chemopotentiators where minimal therapeutic activity is observed alone but in combination with other therapeutics unique drug a more than additive or synergistic improvement in efficacy is observed. General examples include: amonafide with cisplatin or 5-FU. Specific inventive examples for bisbenzylisoquinolines such as 6,6′,7,12-tetramethoxy-2,2′-dimethyl-berbaman (tetrandrine) and derivatives include: signal transduction inhibitors; tyrosine kinase inhibitors such as imatinib, dasatinib, nilotinib and bosutinib; topoisomerase inhibitors; fraudulent nucleosides; fraudulent nucleotides; thymidylate synthase inhibitors; cisplatin or platinum analogs; amino acid substituted alkylating agents such as the nitrosoureas (BCNU, Gliadel wafers, CCNU), bendamustine (Treanda) Temozoloimide (Temodar); anti-tubulin agents; antimetabolites; berberine; apigenin; amonafide; colchicine and analogs; genistein; etoposide; cytarabine; campothecins; vinca alkaloids; topoisomerase inhibitors; 5-fluorouracil; gemcitabine; curcumin; NFKappaB inhibitors; rosmarinic acid; biological therapies such as antibodies such as Avastin, Rituxan, Herceptin, Erbitux; PD-1 and PD-L1 inhibitors; prendimustine; DNA and RNA therapeutics; Braf inhibitors; BTK inhibitors; 5-azacytidine; decitabine; indoleamine 2,3-dioxygenase; daunorubicin; doxycycline; cytarabine; liposomal formulations of daunorubicin and cytarabine; doxorubicin; checkpoint inhibitors; CAR-T cells; indoleamine 2,3-dioxygenase.

Post-treatment patient management: Improvements for suboptimal therapeutics including bisbenzylisoquinolines such as 6,6′,7,12-tetramethoxy-2,2′-dimethyl-berbaman (tetrandrine) and derivatives are made by drugs, treatments and diagnostics to allow for the maximum benefit to patients treated with a compound. General examples include: pain management, nutritional support, anti-emetics, anti-nausea therapies, anti-anemia therapy, anti-inflammatoires. Specific inventive examples for bisbenzylisoquinolines such as 6,6′,7,12-tetramethoxy-2,2′-dimethyl-berbaman (tetrandrine) and derivatives include: use with therapies associated with pain management; nutritional support; anti-emetics; anti-nausea therapies; anti-anemia therapy; anti-inflammatoire antipyretics; immune stimulants; immune modulants.

Alternative medicine/therapeutic support: Improvements for suboptimal therapeutics including bisbenzylisoquinolines such as 6,6′,7,12-tetramethoxy-2,2′-dimethyl-berbaman (tetrandrine) and derivatives are made by the use of unapproved/non-conventional therapeutics or methods to enhance effectiveness or reduce side effects. General examples include: hypnosis, acupuncture, meditation, herbal medications and extracts, applied kinesiology, prayer. Specific inventive examples for bisbenzylisoquinolines such as 6,6′,7,12-tetramethoxy-2,2′-dimethyl-berbaman (tetrandrine) and derivatives include: hypnosis; acupuncture; meditation; herbal medications created either synthetically or through extraction including NF-kappaB inhibitors (such as parthenolide, curcumin, rosmarinic acid); natural antiinflammatories (including rhein, parthenolide); immunostimulants (such as those found in Echinacea); antimicrobials (such as berberine); flavonoids and flavones (such as apigenin, genistein); applied kinesiology; energy medicine; photobiomodulation.

Bulk drug product improvements: Improvements for suboptimal therapeutics including bisbenzylisoquinolines such as 6,6′,7,12-tetramethoxy-2,2′-dimethyl-berbaman (tetrandrine) and derivatives are made by alterations in the pharmaceutical bulk substance. General examples include: salt formation, homogeneic crystalline structure, pure isomers. Specific inventive examples for bisbenzylisoquinolines such as 6,6′,7,12-tetramethoxy-2,2′-dimethyl-berbaman (tetrandrine) and derivatives include: salt formation; homogeneic crystalline structure; pure isomers; increased purity; lower residual solvents and heavy metals.

Diluent systems: Improvements for suboptimal therapeutics including bisbenzylisoquinolines such as 6,6′,7,12-tetramethoxy-2,2′-dimethyl-berbaman (tetrandrine) and derivatives are made by alterations in the diluents used to solubilize and deliver/present the compound for administration. General examples include: Cremophor-EL, cyclodextrins for poorly water soluble compounds. Specific inventive examples for bisbenzylisoquinolines such as 6,6′,7,12-tetramethoxy-2,2′-dimethyl-berbaman (tetrandrine) and derivatives include: use of emulsions; DMSO; NMF; DMF; DMA; ethanol; benzyl alcohol; dextrose containing water for injection; Cremophor; cyclodextrins; PEG.

Solvent systems: Improvements for suboptimal therapeutics including bisbenzylisoquinolines such as 6,6′,7,12-tetramethoxy-2,2′-dimethyl-berbaman (tetrandrine) and derivatives are made by alterations in the solvents used or required to solubilize a compound for administration or for further dilution. General examples include: ethanol, dimethyl acetamide (DMA). Specific inventive examples for bisbenzylisoquinolines such as 6,6′,7,12-tetramethoxy-2,2′-dimethyl-berbaman (tetrandrine) and derivatives include: the use of emulsions; DMSO; NMF; DMF; DMA; ethanol; benzyl alcohol; dextrose containing water for injection; Cremophor; PEG.

Excipients: Improvements for suboptimal therapeutics including bisbenzylisoquinolines such as 6,6′,7,12-tetramethoxy-2,2′-dimethyl-berbaman (tetrandrine) and derivatives are made by alterations in the materials/excipients, buffering agents, preservatives required to stabilize and present a chemical compound for proper administration. General examples include: mannitol, albumin, EDTA, sodium bisulfite, benzyl alcohol. Specific inventive examples for bisbenzylisoquinolines such as 6,6′,7,12-tetramethoxy-2,2′-dimethyl-berbaman (tetrandrine) and derivatives include: the use of mannitol; albumin; EDTA; sodium bisulfite; benzyl alcohol; carbonate buffers; phosphate buffers.

Dosage forms: Improvements for suboptimal therapeutics including bisbenzylisoquinolines such as 6,6′,7,12-tetramethoxy-2,2′-dimethyl-berbaman (tetrandrine) and derivatives are made by alterations in the potential dosage forms of the compound dependent on the route of administration, duration of effect, plasma levels required, exposure to side-effect normal tissues and metabolizing enzymes. General examples include: tablets, capsules, topical gels, creams, patches, suppositories. Specific inventive examples for bisbenzylisoquinolines such as 6,6′,7,12-tetramethoxy-2,2′-dimethyl-berbaman (tetrandrine) and derivatives include: the use of tablets; capsules; topical gels; topical creams; patches; suppositories; lyophilized dosage fills.

Dosage kits and packaging: Improvements for suboptimal therapeutics including bisbenzylisoquinolines such as 6,6′,7,12-tetramethoxy-2,2′-dimethyl-berbaman (tetrandrine) and derivatives are made by alterations in the dosage forms, container/closure systems, accuracy of mixing and dosage preparation and presentation. General examples include: amber vials to protect from light, stoppers with specialized coatings. Specific inventive examples for bisbenzylisoquinolines such as 6,6′,7,12-tetramethoxy-2,2′-dimethyl-berbaman (tetrandrine) and derivatives include: the use of amber vials to protect from light; stoppers with specialized coatings to improve shelf-life stability.

Drug delivery systems: Improvements for suboptimal therapeutics including bisbenzylisoquinolines such as 6,6′,7,12-tetramethoxy-2,2′-dimethyl-berbaman (tetrandrine) and derivatives are made by the use of delivery systems to improve the potential attributes of a pharmaceutical product such as convenience, duration of effect, reduction of toxicities. General examples include: nanocrystals, biocrodible polymers, liposomes, slow release injectable gels, microspheres. Specific inventive examples for bisbenzylisoquinolines such as 6,6′,7,12-tetramethoxy-2,2′-dimethyl-berbaman (tetrandrine) and derivatives include: the use of nanocrystals; bioerodible polymers; liposomes; slow release injectable gels; microspheres.

Drug conjugate forms: Improvements for suboptimal therapeutics including bisbenzylisoquinolines such as 6,6′,7,12-tetramethoxy-2,2′-dimethyl-berbaman (tetrandrine) and derivatives are made by alterations to the parent molecule with covalent, ionic, hydrogen bonded moieties to alter the efficacy, toxicity, pharmacokinetics, metabolism, or route of administration. General examples include: polymer systems such as polyethylene glycols, polylactides, polyglycolides, amino acids, peptides, multivalent linkers. Specific inventive examples for bisbenzylisoquinolines such as 6,6′,7,12-tetramethoxy-2,2′-dimethyl-berbaman (tetrandrine) and derivatives include: the use of polymer systems such as polyethylene glycols; polylactides; polyglycolides; amino acids; peptides; multivalent linkers.

Compound analogs: Improvements for suboptimal therapeutics including bisbenzylisoquinolines such as 6,6′,7,12-tetramethoxy-2,2′-dimethyl-berbaman (tetrandrine) and derivatives are made by alterations to the parent structure of a molecule with additional chemical functionalities that may alter efficacy, or reduce toxicity, pharmacological performance, route of administration, etc. General examples include: alteration of side chains to increase or decrease lipophilicity, additional chemical functionalities to alter reactivity, electron affinity, binding capacity, salt forms. Specific inventive examples for bisbenzylisoquinolines such as 6,6′,7,12-tetramethoxy-2,2′-dimethyl-berbaman (tetrandrine) and derivatives include: alteration of side chains to increase or decrease lipophilicity; additional chemical functionalities to alter reactivity; electron affinity; binding capacity; salt forms; alteration of side chains to increase solubility.

Prodrug systems: Improvements for suboptimal therapeutics including bisbenzylisoquinolines such as 6,6′,7,12-tetramethoxy-2,2′-dimethyl-berbaman (tetrandrine) and derivatives are made by alterations to the molecule such that improved pharmaceutical performance is gained with a variant of the active molecule in that after introduction into the body a portion of the molecule is cleaved to reveal the preferred active molecule. General examples include: enzyme sensitive esters, dimers, Schiff bases. Specific inventive examples for bisbenzylisoquinolines such as 6,6′,7,12-tetramethoxy-2,2′-dimethyl-berbaman (tetrandrine) and derivatives include: the use of enzyme sensitive esters; dimers; Schiff bases; pyridoxal complexes; caffeine complexes.

Multiple drug systems: Improvements for suboptimal therapeutics including bisbenzylisoquinolines such as 6,6′,7,12-tetramethoxy-2,2′-dimethyl-berbaman (tetrandrine) and derivatives are made by the use of additional compounds, biological agents that when administered in the proper fashion, a unique and beneficial effect can be realized. General examples include: inhibitors of multi-drug resistance, specific drug resistance inhibitors, specific inhibitors of selective enzymes, signal transduction inhibitors, repair inhibition. Specific inventive examples for bisbenzylisoquinolines such as 6,6′,7,12-tetramethoxy-2,2′-dimethyl-berbaman (tetrandrine) and derivatives include: the use of inhibitors of multi-drug resistance; specific drug resistance inhibitors; specific inhibitors of selective enzymes; signal transduction inhibitors; tyrosine kinase inhibitors; DNA repair inhibition; topoisomerase inhibitors with non-overlapping side effects; biological agents with inhibitory effects.

Biotherapeutic enhancement: Improvements for suboptimal therapeutics including bisbenzylisoquinolines such as 6,6′,7,12-tetramethoxy-2,2′-dimethyl-berbaman (tetrandrine) and derivatives are made by its use in combination as sensitizers/potentiators with biological response modifiers. General examples include: use in combination as sensitizers/potentiators with biological response modifiers, cytokines, lymphokines, therapeutic antibodies, antisense therapies, gene therapies. Specific inventive examples for bisbenzylisoquinolines such as 6,6′,7,12-tetramethoxy-2,2′-dimethyl-berbaman (tetrandrine) and derivatives include: use in combination as sensitizers/potentiators with biological response modifiers; cytokines; lymphokines; therapeutic antibodies; antisense therapies such as Avastin, Herceptin, Rituxan, and Erbitux; gene therapies; ribozymes; RNA interference; cell based therapeutics.

Biotherapeutic resistance modulation: Improvements for suboptimal therapeutics including bisbenzylisoquinolines such as 6,6′,7,12-tetramethoxy-2,2′-dimethyl-berbaman (tetrandrine) and derivatives are made by exploiting their selective use to overcome developing or complete resistance to the efficient use of biotherapeutics. General examples include: tumors resistant to the effects of biological response modifiers, cytokines, lymphokines, therapeutic antibodies, antisense therapies, gene therapies. Specific inventive examples for bisbenzylisoquinolines such as 6,6′,7,12-tetramethoxy-2,2′-dimethyl-berbaman (tetrandrine) and derivatives include: the use against tumors resistant to the effects of biological response modifiers; cytokines; lymphokines; therapeutic antibodies; antisense therapies such as Avastin, Rituxan, Herceptin, Erbitux; gene therapies; ribozymes; RNA interference; cell based therapeutics, CAR-T cells.

Radiation therapy enhancement: Improvements for suboptimal therapeutics including bisbenzylisoquinolines such as 6,6′,7,12-tetramethoxy-2,2′-dimethyl-berbaman (tetrandrine) and derivatives are made by exploiting their use in combination with ionizing radiation, phototherapies, heat therapies, radio-frequency generated therapies. General examples include: hypoxic cell sensitizers, radiation sensitizers/protectors, photosensitizers, radiation repair inhibitors. Specific inventive examples for bisbenzylisoquinolines such as 6,6′,7,12-tetramethoxy-2,2′-dimethyl-berbaman (tetrandrine) and derivatives include: the use with hypoxic cell sensitizers; radiation sensitizers/protectors; photosensitizers; radiation repair inhibitors; thiol depletion; vaso-targeted agents; use with radioactive seeds, radionuclides, radiolabeled antibodies, brachytherapy.

Novel mechanisms of actions: Improvements for suboptimal therapeutics including bisbenzylisoquinolines such as 6,6′,7,12-tetramethoxy-2,2′-dimethyl-berbaman (tetrandrine) and derivatives are made by optimizing their utility by determining the various mechanisms of actions, biological targets of a compound for greater understanding and precision to better exploit the utility of the molecule. General examples include: imatinib for chronic myelogenous leukemia (CML), arsenic trioxide for acute promyelocytic leukemia (APL), retinoic acid for APL. Specific inventive examples for bisbenzylisoquinolines such as 6,6′,7,12-tetramethoxy-2,2′-dimethyl-berbaman (tetrandrine) and derivatives include: the use with inhibitors of poly-ADP ribose polymerase; agents that effect vasculature; vasodilation; oncogenic targeted agents; signal transduction inhibitors; tyrosine kinase inhibitors such as imatinib dasatinib, nilotinib, bosutinib; EGFR inhibition; Protein Kinase C inhibition; Phospholipase C down regulation; jun down regulation; histone genes; VEGF; ornithine decarboxylase; JAK2, jun D; v-jun; GPCR's; protein kinase A; telomerase; prostate specific genes; protein kinases; histone deacetylase; beta-catenin inhibitors; calmodulin antagonists; CaMKII-gamma inhibitors.

Selective target cell population therapeutics: Improvements for suboptimal therapeutics including bisbenzylisoquinolines such as 6,6′,7,12-tetramethoxy-2,2′-dimethyl-berbaman (tetrandrine) and derivatives are made by more precise identification and exposure of the compound to those select cell populations where the compounds effect can be maximally exploited. General examples include: tirapazamine and mitomycin c for hypoxic cells, vinca alkaloids for cells entering mitosis. Specific inventive examples for bisbenzylisoquinolines such as 6,6′,7,12-tetramethoxy-2,2′-dimethyl-berbaman (tetrandrine) and derivatives include: use against radiation sensitive cells; radiation resistant cells; energy depleted cells; endothelial cells; cancer stem cells; immunotherapy resistant cells; cells overexpressing beta-catenin; cells overexpressing CaMKII-gamma.

### Experimental Evidence of ES-3000′s Efficacy in Human Leukemias

### Experiment 1: Effects of Tetrandrine of β-catenin in Human Leukemia (K562) cells

TET's ability in an in vitro assay to reduce β-catenin expression was tested in human erythroleukemic (K562) cells. The first portion of the study consisted of cell exposure to Tetrandrine and preparation of cell lysates. Testing was conducted by Applied Immunology, Menlo Park, CA.

Briefly, the method for the first portion of the study was as follows: K562 cells were grown and maintained in medium (RPMI, 10% FBS, Pen Strep and glutamate). Cells were then exposed to TET, 0, 0.1, 1, 5, 10, 20 & 40 μM for 24 hours at 37° C. Following drug exposure, cell viability was checked using trypan blue, followed by cell lysis. Supernatants were collected and test for protein concentration using a Pierce BCA kit. The protein assay tubes were read on a Spectramax at 562 nm and Softmax@ software was used for protein concentration analysis. Using 8×10⁶ K562 cells per drug concentration, the protein concentration in the cell lysate was determined to be about 1 mg/ml by BCA protein assay as seen in Table 1.

TABLE 1
Effect of Tetrandrine on protein concentration in K562 Cells

| | |
|---|---|
| Treatment | Protein Recovered |

| | | |
|---|---|---|
| 40 | μM | 0.9 mg |
| 20 | μM | 1.0 mg |
| 10 | μM | 1.1 mg |
| 5 | μM | 1.0 mg |
| 1 | μM | 1.1 mg |
| 0.1 | μM | 1.1 mg |
| 0 | μM | 1.2 mg |

| | |
|---|---|
| Cells alone | 1.2 mg |

### Experiment II Effects of Tetrandrine on Beta-Catenin and Beta-Actin Levels in Human Myelogenous Leukemia K562 Cell Line

The second portion was to quantitate the levels of β-catenin protein by western blot. TET's ability in an in vitro assay to reduce β-catenin expression was tested in human erythroleukemic (K562) cells. Testing was conducted by Alamo Laboratories, San Antonia, TX.

Briefly, the method to quantitate β-catenin protein by western blot was as follows: Extracts of K562 cells (untreated and treated with various concentrations of tetrandrine) were concentrated to 0.4 ml using Centricon-3 column to yield a protein concentration of approximately 2 mg/ml. Samples were mixed with 5× Laemmli buffer and loaded on 8-16% Tris-Glycine gradient gel, then polyacrylamide gel electrophoresis (PAGE) was run at 85 volts for 100 minutes at 6° C. and proteins were transferred on PVDF membrane at 70 Volts for 85 minutes at 8° C. Membrane was blocked with 5% milk in Tris-buffered Saline containing 0.1% TW-20 (TBST). Membrane was incubated with primary antibody for 18 hours at 4° C. followed by 3 washes, 5 minutes each, with TBST and incubated with secondary antibody in TBST for 2 hours at 25° C. The membrane was washed again with TBST as described above and incubated with ECL-2 substrate for 5 minutes and scanned on Kodak Image Station. After scan, blot was stripped of primary and secondary antibodies and re-probed with anti-beta Actin antibody as described above. Protein bands in WB images were quantitated using Molecular Imaging software pack MIS-4.0 software and tabulated in Excel spreadsheet.

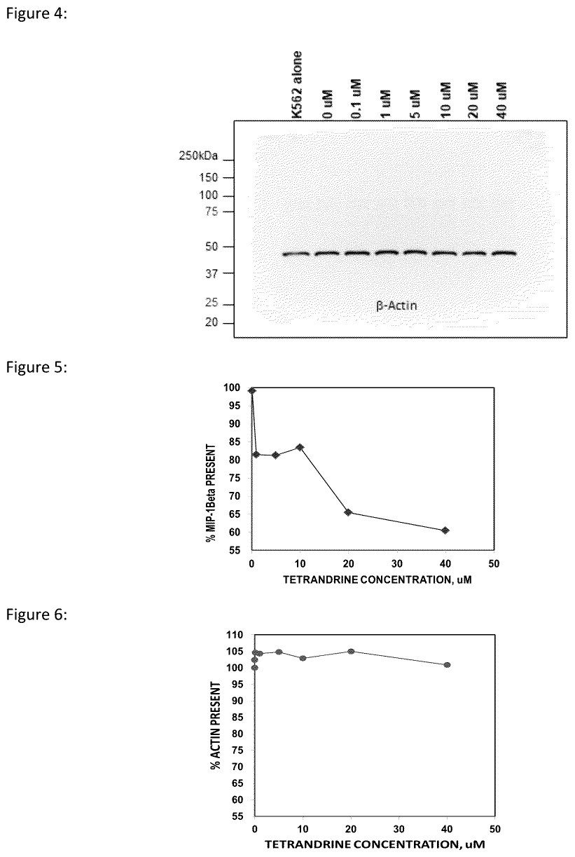

Results indicated that Tetrandrine lowers the β-catenin level in K562 cells in a dose dependent manner as seen in Table 2. It is also important to note the β-catenin protein exists as a doublet in all K562 cell extracts, including untreated cells. The doublet could arise from proteolysis, posttranslational modification or mRNA splicing. A clear β-catenin band was evident in extracts of K562 cells treated with 0 to 10 uM tetrandrine (FIG. 3). However, a convincing β-catenin band was absent in K562 cells treated with 20 and 40 uM tetrandrine. Also, a clear beta-Actin band was evident in all extracts of K562 cells treated with 0 to 40 uM tetrandrine (FIG. 4). Quantitation of the protein-bands in FIGS. 1 and 2, revealed that beta-catenin level was significantly less in extracts of K562 cells treated with >10 uM tetrandrine (FIG. 5); whereas the beta-Actin level in extracts of K562 cells treated with up to 40 uM tetrandrine was not affected significantly by drug treatment (FIG. 6). We conclude that TET reduces β-catenin expression in K562 Cells.

The effect of TET treatment on K562 percent cell viability is depicted in Table 2 and is graphically illustrated in FIG. 2.

TABLE 2
Effect of TET on K562 cell viability

| | |
|---|---|
| Treatment | % Viability |

| | | |
|---|---|---|
| 40 | μM | 56.4 |
| 20 | μM | 76.5 |
| 10 | μM | 79.7 |
| 5 | μM | 81.8 |
| 1 | μM | 79.2 |
| 0.1 | μM | 82.4 |
| 0 | μM | 81.1 |

| | |
|---|---|
| Cells alone | 86.7 |

### Experiment III: Effects of Tetrandrine in Leukemia Patient Derived Cells (In Vitro)

Study Title: In Vitro Evaluation of One Test Compound on Human Chronic Myeloid Leukemia using Colony Forming Cell Assays
Study Report: END 01
Testing Entity: Reach Bio, Seattle, WA

An in vitro investigation was performed to determine whether TET has efficacy on human chronic myeloid leukemia (CML) stem-like cells. A colony forming cell (CFC) assay was performed on bone marrow cells from a de-novo CML patient.

Human bone marrow light density cells derived from CML patient bone marrow (Conversant Bio, Alabama), were stored in the gaseous phase of liquid nitrogen until required. On the day of the experiment, the cells were thawed rapidly, the contents of the vial were diluted in 10 mL of Iscove's Modified Dulbecco's Medium containing 10% fetal bovine serum (IMDM+10% FBS) and washed by centrifugation (approximately 1200 r.p.m. for 10 minutes, room temperature). The supernatant was discarded and the cell pellets resuspended in a known volume of IMDM+10% FBS. A cell count (3% glacial acetic acid) and viability assessment (trypan blue exclusion test) were treated with Tetrandrine (10 μM) for 14 days. After treatment, primary and secondary colonies were grown and analyzed by qPCR to determine BCR-ABL or ABL only cells.

Human erythroid (CFU-E and BFU-E) and myeloid (CFU-GM) colony enumeration were performed by trained personnel. In addition, the distribution of colony types as well as general colony and cellular morphology were analyzed. The variance in colony number detected in replicate cultures was representative of the historical coefficient of variation for colony enumeration using these types of assays. The number and distribution of colonies detected in the solvent control (0.2% DMSO) was not statistically different from the standard control (containing neither Tetrandrine or Imatinib compounds nor DMSO). For statistical analysis, colony numbers in compound-treated cultures were compared to the solvent control cultures. Potential effects of Tetrandrine and CML progenitors are presented in Table 3.

TABLE 3
Effect of ES-3000 (Tetrandrine) and Imatinib
on CML in a Methylcellulose-based Medium

| | | | Total | | CFU- | |
|---|---|---|---|---|---|---|
| | CFU-E | BFU-E | Erythroid | CFU-GM | GEMM | Total CFC |
| Standard | 10 ± 3 | 23 ± 2 | 33 ± 4 | 60 ± 4 | ND | 93 ± 0 |
| Solvent Control | 10 ± 1 | 23 ± 4 | 33 ± 4 | 56 ± 5 | ND | 89 ± 7 |
| ES-3000 30 μM | ND**** | ND** | ND** | ND** | ND | ND*** |
| ES-3000 10 μM | ND**** | ND** | ND** | 26 ± 2* | ND | 26 ± 2*** |
| ES-3000 3 μM | 9 ± 2 | 14 ± 3 | 23 ± 2 | 52 ± 5 | ND | 75 ± 6 |
| ES-3000 1 μM | 10 ± 2 | 22 ± 4 | 33 ± 3 | 54 ± 2 | ND | 86 ± 4 |
| ES-3000 0.1 μM | 11 ± 1 | 22 ± 4 | 33 ± 3 | 55 ± 5 | ND | 88 ± 8 |
| Imatinib 30 μM | ND**** | ND** | ND** | 5 ± 1** | ND | 5 ± 1*** |
| Imatinib 10 μM | ND**** | ND** | ND** | 12 ± 2** | ND | 12 ± 2*** |
| Imatinib 3 μM | ND**** | ND** | ND** | 27 ± 6 | ND | 27 ± 6** |
| Imatinib 1 μM | ND**** | ND** | ND** | 39 ± 6 | ND | 39 ± 6** |
| Imatinib 0.1 μM | 9 ± 1 | 18 ± 4 | 28 ± 5 | 57 ± 3 | ND | 85 ± 7 |

*p < 0.01;
**p < 0.001;
***p < 0.0001;
****p < 0.00001;
ND none detected

Of interest, the media formulation used for the culture of the progenitors, did not contain erythropoietin so the erythroid colonies which were detected in these cultures represent Epo independent clonal growth. ES-3000 had significant effect on CFU-GM colonies at 30 and 10 μM, while also having the same effect towards the erythroid lineage. The experimental positive control, Imatinib, also had a significant effect on CFU-GM progenitors at 30 and 10 μM but was more toxic to the erythroid lineage (30 to 1 μM) when compared to ES-3000. IC₅₀ values were determined and based on these, Imatinib was more toxic than ES-3000, Table 4.

TABLE 4
Effects of ES-3000 (Tetrandrine) and Imatinib
on Human Progenitors CML marrow

| | Erythroid IC50 ± 95% | Myeloid IC50 ± 95% |
|---|---|---|
| Compound | CI (μM) | CI (μM) |
| ES-3000 | 3.12 ± 0.01 | 9.56 ± 1.25 |
| Imatinib | 0.12 ± 0 | 2.66 ± 0.86 |

### Experiment IV: Replating Assay

This CML sample supported secondary clonal growth as determined through the replating assay (Table 3). In the solvent control cultures, 66.7% wells supported secondary growth with one or more colony and an average of three CFC per well. In the cultures initiated with colonies derived from the original cultures with Imatinib @ 3 μM, 16.7% of wells supported secondary growth with one or more colony and an average of two CFC per well. In the cultures initiated with colonies derived from the original cultures with ES-3000 @10 μM, 54.2% of wells supported secondary growth with one or more colony and an average of three CFC per well.

### Experiment V: qPCR Assay for bcr-abl

To determine whether a colony was normal (bcr-abl negative and abl positive) or diseased (bcr-abl and abl positive), a TaqMan®-based qPCR test was used. In order to score the colonies positive or negative, the following scoring strategy was performed. All colonies were assayed in duplicate for each target. If a singleton reaction yielded a Ct then the reaction scored a 1. If no Ct was recorded, a score of 0 was given. From here, we used a logic test to score the colonies into four different categories: bcr-abl and abl, abl only, bcr-abl only or both negative. In order to be placed in a category, a colony must score a 1 in both reactions. However, if a colony only scored a 1 for one out of the two reactions, this colony was removed from the analysis. If a colony scored a 1 for each reaction (both ber-abl and abl) the colony was placed into the ber-abl and abl category, signifying it as a diseased colony. Alternatively, if a colony scored a 1 in only both of the ber-abl reactions or a 1 in only both the abl reactions, the colony was placed in the ber-abl only or abl only category, respectively. If the colony scored a 0 for both targets (bcr-abl and abl) then the colony was placed in the both negative category.

In total 48 primary colonies and 31 secondary replated colonies were assessed for bcr-abl and abl using qPCR. Using the scoring logic described above, three colonies were removed from the analysis. Thus, this yielded 20 out of 21 colonies (95.2%) scoring positive for both bcr-abl and abl. Seven colonies treated with Tetrandrine at 10 μM were removed from the analysis leaving 11 out of 17 colonies bcr-abl and abl positive (64.7%), One out of 17 was abl positive only (5.8%), while one out of 17 was bcr-abl positive only (5.8) and 4 out of 17 colonies were negative for both targets (23.5%). Most of the secondary colonies treated in solvent control conditions score positive for both bcr-abl and abl (10 out of 14, 71.4%) While one out of 14 (7.1%) was positive for abl only and one out of 14 (7.1%) was positive of bcr-abl only. Two out of 14 colonies scored negative for both targets. Thirteen secondary colonies treated with Tetrandrine at 10 μM were assessed. However, most colonies scored negative for both bcr-abl and abl (9 out of 10, 90%) suggesting the colony's transcript level was out of the level of detection of the assay. Only 1 out of 10 colonies scored positive for abl only (10%). No colonies scored positive for bcr-abl and abl. Further, a few secondary colonies that were treated with Imatinib at 3 μM were assessed for bcr-abl and abl. Only one out of four colonies was positive for both targets, while the remaining three colonies were negative for both targets. The results are provided in Tables 5 and 6. Table 5 indicates that in the solvent control cultures, 66.7% wells supported secondary growth with one or more colony and an average of three CFC per well. In the cultures initiated with colonies derived from the original cultures with Imatinib @ 3 μM, 16.7% of wells supported secondary growth with one or more colony and an average of two CFC per well. In the cultures initiated with colonies derived from the original cultures with ES-3000 @10 μM, 54.2% of wells supported secondary growth with one or more colony and an average of three CFC per well.

TABLE 5
Repeating Colony Count

| Solvent Control | | | | Imatinib @, 3 μM | | | | ES-3000 @ 10 μM | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | | | |
|---|---|---|---|---|---|---|---|---|---|---|---|---|
| A | 3 | 4 | 3 | 3 | 0 | 0 | 0 | 1 | 0 | 0 | 5 | 0 |
| B | 1 | 2 | 1 | 1 | 0 | 0 | 2 | 3 | 0 | 0 | 0 | 2 |
| C | 4 | 0 | 7 | 0 | 0 | 0 | 0 | 0 | 1 | 2 | 5 | 1 |
| D | 4 | 0 | 0 | 1 | 0 | 0 | 0 | 0 | 1 | 0 | 0 | 1 |
| E | 0 | 0 | 0 | 4 | 1 | 0 | 0 | 0 | 1 | 6 | 7 | 2 |
| F | 5 | 1 | 0 | 3 | 0 | 0 | 0 | 0 | 0 | 0 | 0 | 2 |

TABLE 6
bcr-abl Colony Assessment Using qPCR

| | | bcr-abl | | | | | Total |
|---|---|---|---|---|---|---|---|
| | | and abl | | bcr-abl | both | | Colonies |
| | | colonies | abl only | only | negative | Omit | Assessed |
| Primary | Solvent Control | 20 | 0 | 0 | 1 | 3 | 24 |
| | ES - 3000 @ 10 μM | 11 | 1 | 1 | 4 | 7 | 24 |
| Secondary | Solvent Control | 10 | 1 | 1 | 2 | 0 | 14 |
| | ES - 3000 @, 10 μM | 0 | 1 | 0 | 9 | 3 | 13 |
| | Imatinib @ 3 μM | 1 | 0 | 0 | 3 | 0 | 4 |

In conclusion, a semi-solid culture system which supports the proliferation of chronic myeloid progenitors, test compound ES-3000 was assessed and compared with Imatinib on primary and secondary clonal growth. This patient sample supported Epo independent growth (i.e. erythroid colonies were detected in the cultures though Epo was not added to the medium). ES-3000 and Imatinib both inhibited erythroid and myeloid colonies, though the IC50 values for Imatinib were lower than those for ES-3000. Secondary cultures were assessed by plucking individual colonies from the solvent control cultures as well as those from cultures to which Imatinib at 3 μM and ES-3000 at 10 μM were added (representing their respective IC50 values and replating them in fresh culture medium. In the solvent control cultures, 66.7% of colonies supported secondary growth as compared to 16.7% of the Imatinib treated cultures and 54.2% of the ES-3000 treated cultures. A qPCR assay was used to detect bcr-abl transcripts in plucked colonies. 95 percent of colonies from solvent control cultures were bcr-abl positive compared to 65 percent of colonies treated with ES-3000. Upon analyzing colonies from the secondary replating, most colonies treated with ES-3000 were below the limit of detection while a majority solvent control colonies remained bcr-abl positive.

### Experiment VI: Effect of Tetrandrine in a Leukemia Mouse Model

Study Title: Inhibition of CML Stem Cells with an Alkaloid that Reduces β-catenin
Study Report: In Vivo Report UMass
Testing Entity: University of Massachusetts Medical School (Dr. Li's Lab), Worchester, MA

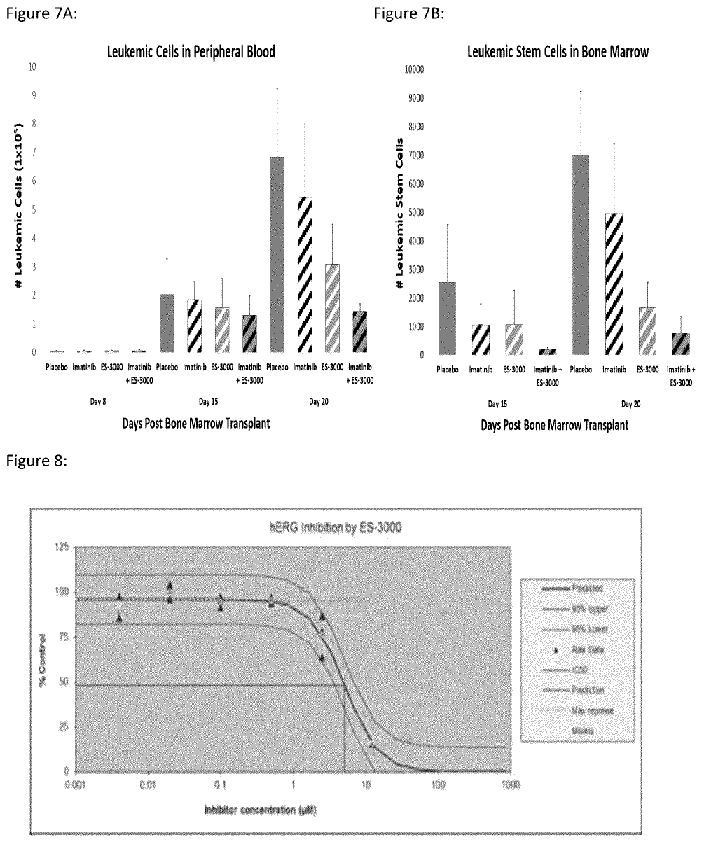

In an in vivo model, the effectiveness of Tetrandrine was tested on CML stem cells. The objective of the study was to determine the survival of leukemic cells and leukemic stem cells in response to the treatment with Tetrandrine. After priming donor C57BL/6 (B6) mice with intravenous injections of 5-fluorouracil for four days, bone marrow cells were harvested from femurs and tibia, then transfected twice with retrovirus containing MSCV-BCR-ABL-IRES-GFP. Recipient mice were lethally irradiated by two doses of 550 cGy before bone marrow transplantation by intravenous injection with 5×10⁵ cells/mouse. Blood from recipient mice was tested for disease induction one week after transduction by FACS analysis for GFP (FIG. 7A). All mice tested positive. Treatments started on day 8 after bone marrow transplantation (FIG. 7B). Mice were randomized into four groups and treated orally with vehicle [3×/day, 2×], Imatinib [100 mg/kg, 2×/day], Tetrandrine [150 mg/kg, 1×/day] or Imatinib+Tetrandrine [3×/day: 2× with Imatinib, 1× with Tetrandrine].

The results of this study demonstrated that Tetrandrine given orally once a day is superior to Imatinib given twice a day in inhibiting the development of both circulating leukemic cells and leukemic stem cells while the combination of Tetrandrine with Imatinib further improves efficacy of Tetrandrine.

### Experiment VII: Effect of Tetrandrine on CamKIlγ Activity

Study Title: ES-3000 inhibition of CAMKII gamma
Study Report: CamKII Activity Report ES-3000
Testing Entity: Institute for Rare and Neglected Diseases Drug Discovery , Mountain View, CA

An enzymatic study was conducted to evaluate the inhibition of CAMKII gamma by ES-3000 (Tetrandrine). CAMKII gamma kinase activity was measured using ATP GLO purchased from Promega following the Promega protocol. CAMKII enzyme system purchased from Promega (V3531). ADP Glo was used for detection. Assay optimized for linearity of protein and time. Conditions for inhibitor screening 25 ng CAMKII, 20 min. incubations, inhibitor or DMSO (5%) at RT in 20 uL total volume. Inhibitor, ATP (10 or 50 uM), substrates and buffer added to tubes and reactions started with enzyme. Staurosporin was used as positive control. Reactions terminated at 20 min. by adding ADP Glo reaction following manufactures instructions. All reactions were done in duplicate. Calculations of percent (%) inhibition determined by dividing the mean activity at each condition by the mean of the DMSO control times 100.

Results of individual experiments with 10 μM or 50 μM ATP are presented in Table 7 and Table 8, respectively.

TABLE 7
CamKIIγ Activity Inhibition by Tetrandrine with 10 uM ATP

| | | | | | % |
|---|---|---|---|---|---|
| Conditions | units | units | mean | % DMSO | inhibition |
| DMSO control | 4.56 | 4.15 | 4.355 | 100% | 0.1 |
| berbamine 250 uM | 2.38 | 2.3 | 2.34 | 54% | 46.3 |
| berbamine 100 uM | 2.29 | 2.32 | 2.305 | 53% | 47.1 |
| berbamine 30 uM | 2.55 | 2.967 | 2.7585 | 63% | 36.7 |
| ES-3000 250 uM | 0.34 | 0.48 | 0.41 | 9% | 90.6 |
| ES-3000 100 uM | 2.5 | 3.24 | 2.87 | 66% | 34.2 |
| ES-3000 30 uM | 2.75 | 3.43 | 3.09 | 71% | 29.1 |
| Staurosporin 10 nM | 0.93 | 0.9 | 0.915 | 21% | 79.0 |
| Staurosporin 100 nM | 0.25 | 0.15 | 0.2 | 5% | 95.4 |
| DMSO control | 4.16 | | 4.16 | 95% | 4.6 |
| ADP std - 10 uM | 0.52 | 0.44 | 0.48 | | |
| ATP std - 10 uM | 20.2 | 22.8 | 21.5 | | |

TABLE 8
CamKIIγ Activity Inhibition by Tetrandrine with 50 uM ATP

| | | | | % | % |
|---|---|---|---|---|---|
| Condition | units | units | mean | DMSO | inhibition |
| ES-3000 100 uM | 8.81 | 7.1 | 7.955 | 64% | 36 |
| ES-3000 30 uM | 10.4 | 10.7 | 10.55 | 85% | 15 |
| Staurosporin 10 nM | 9.93 | 4.64 | 7.285 | 59% | 41 |
| Staurosporin 100 nM | 0.67 | 0.6 | 0.635 | 5% | 95 |
| DMSO control | 11.5 | 13.3 | 12.4 | 1 | |
| ATP std - 10 uM | 0.34 | 0.37 | 0.355 | | |
| ADP std - 10 uM | 9.09 | 7.15 | 8.12 | | |

The results from the study showed a good signal to noise. Staurosporin was effective as a positive control and ES-3000 inhibited CAMKII gamma kinase activity in a dose dependent manner.

TABLE 9
CamKIIγ Activity Inhibition by Tetrandrine

| | Percent inhibition | Percent inhibition |
|---|---|---|
| | 10 uM ATP | 50 uM ATP |
| ES-3000 250 uM | 91 | |
| ES-3000 100 uM | 34 | 36 |
| ES-3000 30 uM | 29 | 15 |
| staurosporin 100 nM | 95 | 95 |
| staurosporin 10 nM | 79 | 41 |
| berbamine 250 uM | 46 | |
| berbamine 100 uM | 47 | |
| berbamine 30 uM | 37 | |

Experiment VIII Effects of TET on hERG
Study Title: hERG Inhibition Potential by the Test Article ES-3000
Study Report: CYP1328_R1
Testing Entity: Cyprotex US, LLC, Watertown, MA

The purpose of this study was to evaluate the potential of a test article to inhibit hERG. The experiments are performed on an Ion Works™ HT instrument (Molecular Devices Corporation), which automatically performs electrophysiology measurements in 48 single cells simultaneously in aspecialized 384-well plate (PatchPlate). All cell suspensions, buffers and test compound solutions are at room temperature during the experiment. The cells used are Chinese hamster ovary (CHO) cells stably transfected with hERG (cell-line obtained from Cytomyx, UK). A single-cell suspension is prepared in extracellular solution (Dulbecco's phosphate buffered saline with calcium and magnesium pH 7-7.2) and aliquots added automatically to each well of a PatchPlate™. The cells are then positioned over a small hole at the bottom of each well by applying a vacuum beneath the plate to form an electrical seal. The vacuum is applied through a single compartment common to all wells which is filled with intracellular solution (buffered to pH 7.2 with HEPES). The resistance of cach seal is measured via a common ground-electrode in the intracellular compartment and individual electrodes placed into each of the upper wells. Electrical access to the cell is then achieved by circulating a perforating agent, amphotericin, underneath the PatchPlate and then measuring the pre-compound hERG current. An electrode is positioned in the extracellular compartment and a holding potential of −80 mV applied for 15 sec. The hERG channels are then activated by applying a depolarizing step to +40 mV for 5 sec and then clamped at −50 mV for 4 sec to elicit the hERG tail current, before returning to −80 mV for 0.3 sec. Test compound is then added automatically to the upper wells of the PatchPlateTM from a 96-well microtitre plate containing a range of concentrations of each compound. Solutions are prepared by diluting DMSO solutions of the test compound into extracellular (final DMSO concentration 0.25%). The test compound is left in contact with the cells for 300 sec before recording currents using the same voltage-step protocol as in the pre-compound scan. Quinidine, an established hERG inhibitor, is included as a positive control and buffer containing 0.25% DMSO is included as a negative control. The results for all compounds on the plate are rejected and the experiment repeated if the IC50 value for quinidine or the negative control results are outside quality-control limits. Each concentration is tested in 4 replicate wells on the PatchPlate™. However, only cells with a seal resistance greater than 50 mOhm and a pre-compound current of at least 0.1 nA are used to evaluate hERG blockade. Post-compound currents are then expressed as a percentage of pre-compound currents and plotted against concentration for each compound (FIG. 8). Testing was conducted by Cyprotex US, LLC, Watertown, MA. Experimental conditions (Table 10) and results (Table 11) are presented below.

TABLE 10
hERG Inhibition Experimental Conditions

| | | Reference | |
|---|---|---|---|
| Test Article | Test Conc. | Compound | Analytical Method |
| ES-3000 | .004-12.5 μM | Quinidine | electrophysiology |

TABLE 11
Results of hERG Inhibition by ES-3000

| | Test Conc. | | | |
|---|---|---|---|---|
| Test Article | (μM) | IC₅₀ (μM) | SE IC₅₀ | Comment |
| Quinidine | 0.01-10 | 1.66 | 0.353 | Positive |
| | | | | Control |
| ES-3000 | 0.004-12.5 | 5.15 | 0.699 | |

Tetrandrine or TET has been shown to have multiple pharmacological activities relating to anticancer functions. Those include inhibition of Wnt/β-catenin, reduction of expression of MDR1 and inhibition of P-gp function, activation of apoptosis, and suppression of proliferation via various activation and survival signaling pathways. We have further investigated some of the pharmacologic properties most relevant to the use of TET for leukemia. Results from our studies demonstrated that TET reduced β-catenin in human (K562) leukemia cells, reduced bcr-able positive primary colonies and eliminated bcr-able positive secondary colonies in a colony forming cell assay. In a murine model of leukemia, TET reduced the development of leukemic cells in the peripheral blood and leukemic stem cells in the bone marrow of mice. In an vitro enzymatic assay, TET inhibited the activation of CaMKIIγ in a dose dependent manner.

In vitro evaluation of TET's ability to inhibit hERG, revealed an IC₅₀ of 5.2 μM, which is 13-fold higher safety for cardiac conduction-related toxicity utilizing Cₘₐₓ from previous human clinical experience [Oldham 1998, Oldham 2000]. Though, QT changes may be observed at concentrations lower than IC₅₀, a reasonable interpretation is that we might expect to see QT prolongation in subjects at plasma levels above 249 ng/mL, as Tetrandrine is highly protein bound.

Additionally, the Wnt/β-catenin pathway has been identified as a new target for AML [Wang 2010] and ES-3000 has shown to reduce β-catenin. More recent studies have shown that TET is able to bind competitively to calmodulin (CaM) and may be a novel CaM antagonist [Ma 2013]. Calmodulin protein dependent kinase (CaMKIIγ) is overexpressed in leukemic stem cells and blast cells, and regulates the Wnt/β catenin and STAT3 pathways [Si 2008]. An evaluation of β-catenin and CaMKIIγ levels in patients to determine a correlation between response and expression levels, may provide valuable information in selecting patients who would benefit most with ES-3000 treatment.

The multiplicity of TET's mechanisms and safety profile provides an opportunity for its development as a therapeutic for relapsed or refractory AML.

### REFERENCES

Arcangeli A, Becchetti A, New trends in cancer therapy: targeting ion channels and transporters. Pharmaceuticals, 2010. 3:1202-1224.
Arcangeli A, Pillozzi S, Becchetti A, Targeting ion channels in leukemias: a new challenge for treatment. Curr Med Chem, 2012. 19(5):683-96.
Fanelli M, Hattinger C M, Vella S, Tayanti E, Michelacci F, Gudeman B, Barnett D, Picci P, Serra M, Targeting ABCB1 and ABCC1 with their specific inhibitor CBT-1 can overcome drug resistance in osteosarcoma. 2016, Curr Canc Drug Targets16:261-274.
Griffiths E A, Gore S D, Hooker C M, et al., Acute myeloid leukemia is characterized by Wnt pathway inhibitor promoter hypermethylation. Leuk & Lymph, 2010. 51(9): 1711-1719.
He B C, Gao J-L, Zhang B-Q, et al. Tetrandrine inhibits Wnt/β-catenin signalling and suppresses tumor growth of human colorectal cancer. Mol Pharmacol, 2011. 79(2):211-9.
Huang Y T, Hong C Y. Tetrandrine. Cardiovascular Drug Rev. 1998. 16, 1-15.
Huber S M, Oncochannels. Cell Calcium, 2013. 53(4):241-55.
Jin H, Li L, Zhong D, Liu J, Chen X, Zheng J. Pulmonary Toxicity and Metabolic Activation of Tetrandrine in CD-1 Mice. Chem. Res. Toxicol. 2011. 24, 2142-2152.
Ju A, Kang Y, et al. Pharmacokinetics, tissue distribution and excretion study of tetrandrine in rats. Journal of Chinese Pharmaceutical Sciences. 2015;24(8).
Kelly R J, Robey R W, Chen C C, Draper D, Luchenko V, Barnett D, Oldham R K, Caluag Z, Frye A R, Steinberg S M, Fojo T, Bates S E, A pharmacodynamic study of the P-glycoprotein antagonist CBT-1 in combination with paclitaxel in solid tumours. 2012, The Oncologist. 17:512.
Lang F, Stoumaras C, Ion channels in cancer: future perspectives and clinical potential. Phil Trans R Soc B, 2014. 369: 20130108.
Liu W, Zhang J, Ying C, et al. Tetrandrine combined with gemcitabine and cisplatin for patients with advanced non-small cell lung cancer improve efficacy. Int J Biomed Sci, 2012. 8(1):28-35.
Ma L, Wang Z, Liu S, et al. Screening calmodulin-binding ligands using intensity-fading matrix-assisted laser desorption/ionization mass spectrometry. Rapid Commun Mass Spectrom, 2013. 27: p. 1527-1534.
Ng L T, Chiang L-C, Lin Y-T, et al. Antiproliferative and apoptotic effects of Tetrandrine on different human hepatoma cell lines. Am J Chin Med, 2006. 34(1): 125-35.
Oldham R K, Reid W K, Barnett D, Phase I study of CBT-1 and Taxol in patients with Taxol resistant cancers. 2000, Cancer Biother Radiopharm. 15(2): 153-159.
Oldham R K, Reid W K, Preisler H D, Barnett D. A phase I and pharmacokinetic study of CBT-1 as a multidrug resistance modulator in the treatment of patients with advanced cancer. Cancer Biother Radiopharm. 1998;13:71-80.
Si J, Collins S J, Activated Ca2+/Calmodulin-dependent protein kinase Ilγ is a critical regulator of myeloid leukemia cell proliferation. Cancer Res, 2008. 68(10):3733-3742.
Simon M, Grandage V L, Linch D C and Khwaja A. Constitutive activation of the Wnt/β-catenin signalling pathway in acute myeloid leukaemia. Oncogene 2005. 24:2410-2420.
Susa M, Choy E, Yang C, Schwab J, Mankin H, Hornicek F, Duan Z. 2010. Multidrug resistance reversal agent, NSC77037, identified with a cell-based screening assay. J Biomol Screen. 15, 287-296.
Wang G, Lemos J R, Tetrandrine: a new ligand to block voltage-dependent Ca2+ and Ca2+-activated K+ channels. Life Sci, 1995. 56(5):295-306.
Wang Y, Krivtsov A, Sinha A, The wnt/β-catenin pathway is required for the development of leukemia stem cells in AML. Science, 2010. March 26; 327(5973):1650-1653.
Wu J-M, Chen Y, Chen J-C, Lin T-Y, Tseng S-H, Tetrandrine induces apoptosis and growth suppression of colon cancer cells in mice. 2010, Cancer Letters. 287:187-195.
Wu K, Zhou M, Wu Q-X, Yuan S-X, Wang D-X, Jin J-L, Huang J, Yang J-Q, Sun W-J, Wan L-H, He B-C, The role of IGFBP-5 in mediating the antiproliferation effects of Tetrandrine in human colon cancer cells. 2015, Int. J Oncology. 46:1203-1213.
Xu W L, Shen H L, Ao Z F, Combination of Tetrandrine as a potential-reversing agent with daunorubicin, etoposide and cytarabine for the treatment of refractory and relapsed acute myelogenous leukemia. Leukemia Res, 2006. 30:407-413.
Xu X H, Gan Y C, Xu G B, Chen T, Shou H, Tang J F, Gu Y, Xu F, Xie Y Y, Zhao X Y, Xu R Z, Tetrandrine citrate eliminates imatinib-resistant chronic myeloid leukemia cells in vitro and in vivo by inhibiting Bcr-Able/B-catenin axis. J Zhejiang University-Sci B (Biomedicine & Biotechnology), 2012 13(11):867-874.
Zeng F D, Shaw D H Jr, Ogilvie R I. 1985. Kinetic Disposition and Hemodynamic Effects of Tetrandrine in Anesthetized Dogs. J Cardiovascular Pharmacol.7(6):1034-1039.
Zhejiang Jinhua Conba Bio-Pharma, Co. Ltd., (Jinhua,China), Tetrandrine Tablet Package Insert 2014.
Zhu X, Sui M, Fan W, In vitro and in vivo characterizations of Tetrandrine on the reversal of p-glycoprotein-mediated drug resistance to paclitaxel. 2005, Anticancer Research. 25:1953-1962.

What is claimed:

1. A method to improve the efficacy and /or reduce the side-effects of a less than efficacious drug therapy or therapeutic agent comprising the steps of:
a. identifying at least one factor and/or at least one parameter associated with the efficacy and/or occurrence of side effect(s) of the drug therapy; and
b. modifying the factor or parameter to improve the efficacy and/or reduce the side effects of the drug therapy; wherein the suboptimally administered drug therapy comprises administration of a bisbenzylisoquinoline such as 6,6′,7,12-tetramethoxy-2,2′-dimethyl-berbaman (aka d-tetrandrine, Tetrandrine, NSC 77037, ES-3000, Tet, TET) and derivatives; and wherein at least one factor and/or at least one parameter is selected from the group consisting of:
i. dose modification
ii. route of administration
iii. schedule of administration
iv. indications for use
v. selection of disease stage
vi. other indications
vii. patient selection
viii. patient/disease phenotype
ix. patient/disease genotype
x. pre/post treatment preparation
xi. toxicity management
xii. pharmacokinetic/pharmacodynamic monitoring
xiii. drug combinations
xiv. chemosensitization
xv. chemopotentiation
xvi. post treatment patient management
xvii. bulk drug product improvements
xviii. diluents
xix. solvents
xx. excipients
xxi. dosage forms
xxii. dosage kits and packaging
xxiii. drug delivery systems
xxiv. drug conjugate forms
xxv. analogs
xxvi. prodrugs
xxvii. multiple drug systems
xxviii. biotherapeutic enhancements
xxix. biotherapeutic resistance modulation
xxx. radiation therapy enhancement
xxxi. mechanisms of action
xxxii. selective target cell population therapeutics, and combinations of two or more thereof.

2. The method of claim 1, wherein the drug therapy is administered to treat a hyperproliferative disease or arthritis or SARS-Cov-19.

3. The method of claim 2 wherein the hyperproliferative disease is cancer.

4. The method of claim 1, wherein the bisbenzylisoquinoline is 6,6′,7, 12-tetramethoxy-2,2′-dimethyl-berbaman.

5. The method of claim 1, wherein bisbenzylisoquinoline is Tetrandrine (d-tetrandrine TET, ES-3000).

6. The method of claim 1, wherein bisbenzylisoquinoline is d-tetrandrine.

7. The method of claim 1, wherein the improvement is made by dose modification-selected from the group consisting of: i.v. infusion for hours to days; biweekly, tri-weekly, monthly administration; dose greater than 0.5-6.0 mg/kg/day; progressive escalation of dosing from 1 mg/kg/day to greater than 4 mg/kg/day based on patient tolerance; doses less than 1 mg/kg/day for greater than 14 days; use of polyamine to modulate metabolism; use of eflornithine to modulate metabolism; selected and intermittent boost dose administrations; bolus single and multiple doses escalating from 1 mg/kg; oral doses below 1 or above 1.9 mg/kg/day.

8. The method of claim 1, wherein the improvement is made by the route of administration selected from a group consisting of: topical; intravesical therapyfor bladder cancer; oral administration; slow release oral delivery; extended-release oral delivery; rectal delivery; intrathecal; intraarterial; continuous infusion; intermittent infusion.

9. The method of claim 1, wherein the improvement is made by the schedule of administration selected from a group consisting of: topical; intravesical therapy for bladder cancer; oral administration; slow-release oral delivery; extended-release oral delivery; rectal delivery; intrathecal; intraarterial; continuous infusion; intermittent infusion.

10. The method of claim 1, wherein the improvement is made by the indications for use selected from a group consisting of: leukemias (acute and chronic, AML, ALL, CLL, CML); myelodysplastic syndrome (MDS); leukemia with MLL translocations (11q23); leukemia enriched in FLT3 or NPM somatic mutations; leukemia with genomic alterations in IDHI or DNMT3A genes; angiogenic diseases; benign prostate hypertrophy; psoriasis; gout; autoimmune conditions; prevention of transplantation rejection; restenosis prevention in cardiovascular disease; mycoses fungoides; use in bone marrow transplantation; as an anti-infective; treatment for AIDS; treatment for lymphoma; mantle cell lymphoma; meningeal leukemia; malignant meningitis; cutaneous T cell lymphoma; Barret's esophagus; anaplastic gliomas; triple negative breast cancer; Braf mutated melanoma; BTK resistant CLL; lymphoma; Chordoma; KRAS mutated colon cancer; RAS mutated lung cancer; pancreatic cancer, MDS AML arthritis, Covid.

11. The method of claim 1, wherein the improvement is applicable to selection of disease stage(s) selected from a group consisting of: chronic and acute myeloid leukemias, pre-leukemic conditions such as myelodysplastic syndrome, localized polyp stage colon cancer; leukoplakia in the oral cavity; angiogenesis inhibition to prevent or limit metastatic spread; chronic myeloid leukemias with measurable minimal residual disease.

12. The method of claim 1, wherein the improvement is applicable to other treatment indication(s) selected from a group consisting of: anti-infectives; antivirals; antibacterials; for pleural effusions; antifungals; antiparasitics; eczema; shingles; condyloma; anti HPV; anti-HSV; carly and late stage MDS (Myelodysplastic syndrome); polycythemia vera (PV), arthritis, Covid.

13. The method of claim 1, wherein the improvement is made regarding patient selection, wherein patient selection is selected from the group consisting of: patients with minimal residual disease or resistant disease; patients with cancer stem cells; patients with disease conditions with high levels of metabolic enzymes, histone deacetylase, protein kinases, ornithine decarboxylase; patients with disease conditions with low levels of metabolic enzymes, histone deacetylase, protein kinases, ornithine decarboxylase; patients with low or high susceptibility to thrombocytopenia, neutropenia; patients intolerant of GI toxicities; over expression of c-Jun or v-Jun, GPCR's and signal transduction proteins, VEGF, prostate-specific genes, protein kinases, telomerase; patients with high expression of beta-catenin in cancerous cells; patients with high expression of activated calmodulin-dependent protein kinase II-gamma (CaMKIIγ) in cancerous cells.

14. The method of claim 1, wherein the improvement is made by determination of patient's or disease's phenotype is selected from a group consisting of: diagnostic tools, techniques, kits and assays to confirm a patient's particular phenotype and for the measurement of minimal residual disease, resistant disease or cancer stem cells, metabolism enzymes and metabolites, histone deacetylase, protein kinases, ornithine decarboxylase, VEGF, prostate specific genes, telomerase, JAK2, jun GPCR's; surrogate compound dosing or low dose drug pre-testing for enzymatic status; beta-catenin; calmodulin dependent protein kinase II-gamma (CaMKII-gamma); phospho-CaMKII-gamma.

15. The method of claim 1, wherein the improvement is made by determination of patient's or disease's genotype is selected from a group consisting of: diagnostic tools, techniques, kits and assays to confirm a patient's particular genotype; gene/protein expression chips and analysis; Single Nucleotide Polymorphisms (SNP's) assessment; SNP's for histone deacetylase; ornithine decarboxylase; S-adenosyl methionine GPCR's; protein kinases; telomerase; VEGF; prostate-specific genes; Jun; identification and measurement of metabolism enzymes and metabolites; mutation in wild type and mutated genes.

16. The method of claim 1, wherein the improvement is made by pre-and/or post-treatment preparation is selected from a group consisting of: tyrosine kinase inhibitors (TKIs); daunorubicin and cytarabine or liposomal formulations of daunorubicin and cytarabine; checkpoint inhibitors such as anti-PD1 antibodies, anti-PD-L1 antibodies; CAR-T cells; etoposide; colchicine or analogs; use of diuretics such as probenecid; uricase; non-oral use of nicotinamide; sustained release forms of nicotinamide; inhibitors of polyADP ribose polymerase; caffeine; leucovorin rescue; infection control; antihypertensives; alteration of stem cell populations; pretreatment to limit or prevent graft vs. host (GVH) cytokine storm reactions; post-treatment with chemotherapeutics; post-treatment with tyrosine kinase inhibitors; post-treatment with immunotherapeutics, antivirals antiCovid anti-arthritics.

17. The method of claim 1, wherein the improvement is made by toxicity management selected from a group consisting of: the use of colchicine or analogs; use of diuretics such as probenecid; use of uricase; non-oral use of nicotinamide; sustained release forms of nicotinamide; use of inhibitors of polyADP-ribose polymerase; use of caffeine; leucovorin rescue; sustained release allopurinol; non-oral use of allopurinol; bone marrow transplant stimulants; blood; platelet infusions; Neupogen; G-CSF; GM-CSF; filgrastim; pain management; anti-inflammatoire; fluids; corticosteroids; insulin control medications; antipyretics; anti-nausea treatments; anti-diarrhea treatment; N-acetyl cysteine; antihistamines, limit or prevent mucositis; limit or prevent GVH reactions; limit or prevent cytokine storm reactions; anti-fungals; sodium thiosulfate; N-acetylcysteine; glutathione; platelet transfusions; hypotensives; hypertensives.

18. The method of claim 1, wherein the improvement is made by pharmacokinetic and/or/pharmacodynamic monitoring selected from a group consisting of: multiple determinations of drug plasma levels; multiple determinations of metabolites in the blood or urine; beta-catenin levels; BK channel levels; gene sequencing; immune effectors; CaMKII-gamma; phospho-CaMKII-gamma.

19. The method of claim 1, wherein the improvement is made by administration of drug combinations of at least two or more drug types selected from a group consisting of: signal transduction inhibitors; tyrosine kinase inhibitors such as imatinib, dasatinib, nilotinib and bosutinib; topoisomerase inhibitors; fraudulent nucleosides; fraudulent nucleotides; thymidylate synthase inhibitors; cisplatin or platinum analogs; amino acid substituted alkylating agents such as the nitrosoureas (BCNU, Gliadel wafers, CCNU), bendamustine (Treanda) Temozoloimide (Temodar); anti-tubulin agents; antimetabolites; berberine; apigenin; amonafide; colchicine and analogs; genistein; ctoposide; cytarabine; campothecins; vinca alkaloids; topoisomerase inhibitors; 5-fluorouracil; gemcitabine; curcumin; NFKappaB inhibitors; rosmarinic acid; biological therapies such as antibodies such as Avastin, Rituxan, Herceptin, Erbitux; PD-1 and PD-L1 inhibitors; prendimustine; DNA and RNA therapeutics; Braf inhibitors; BTK inhibitors; 5-azacytidine; decitabine; indoleamine 2,3-dioxygenase; daunorubicin; doxycycline; cytarabine; liposomal formulations of daunorubicin and cytarabine; doxorubicin; checkpoint inhibitors; CAR-T cells; indoleamine 2,3-dioxygenase.

20. The method of claim 1, wherein the improvement is made by chemosensitization methods selected from a group consisting of: signal transduction inhibitors; tyrosine kinase inhibitors such as imatinib, dasatinib, nilotinib and bosutinib; topoisomerase inhibitors; fraudulent nucleosides; fraudulent nucleotides; thymidylate synthase inhibitors; cisplatin or platinum analogs; amino acid substituted alkylating agents such as the nitrosoureas (BCNU, Gliadel wafers, CCNU), bendamustine (Treanda) Temozoloimide (Temodar); anti-tubulin agents; antimetabolites; berberine; apigenin; amonafide; colchicine and analogs; genistein; etoposide; cytarabine; campothecins; vinca alkaloids; topoisomerase inhibitors; 5-fluorouracil; gemcitabine; curcumin; NFKappaB inhibitors; rosmarinic acid; biological therapies such as antibodies such as Avastin, Rituxan, Herceptin, Erbitux; PD-1 and PD-L1 inhibitors; prendimustine; DNA and RNA therapeutics; Braf inhibitors; BTK inhibitors; 5-azacytidine; decitabine; indoleamine 2,3-dioxygenase; daunorubicin; doxycycline; cytarabine; liposomal formulations of daunorubicin and cytarabine; doxorubicin; checkpoint inhibitors; CAR-T cells; indoleamine 2,3-dioxygenase, hypomethylating agents, 5 azacytidine, decitiabine, cedazuridine, decitiabine combined with cedazuridine (Inqovi) antiarthritic agents, anti-TNF antibodies.

21. The method of claim 1, wherein the improvement is made by chemopotentiation methods selected from a group consisting of: signal transduction inhibitors; tyrosine kinase inhibitors such as imatinib, dasatinib, nilotinib and bosutinib; topoisomerase inhibitors; fraudulent nucleosides; fraudulent nucleotides; thymidylate synthase inhibitors; cisplatin or platinum analogs; amino acid substituted alkylating agents such as the nitrosoureas (BCNU, Gliadel wafers, CCNU), bendamustine (Treanda) Temozoloimide (Temodar); anti-tubulin agents; antimetabolites; berberine; apigenin; amonafide; colchicine and analogs; genistein; etoposide; cytarabine; campothecins; vinca alkaloids; topoisomerase inhibitors; 5-fluorouracil; gemcitabine; curcumin; NFKappaB inhibitors; rosmarinic acid; biological therapies such as antibodies such as Avastin, Rituxan, Herceptin, Erbitux; PD-1 and PD-L1 inhibitors; prendimustine; DNA and RNA therapeutics; Braf inhibitors; BTK inhibitors; 5-azacytidine; decitabine; indoleamine 2,3-dioxygenase; daunorubicin; doxycycline; cytarabine; liposomal formulations of daunorubicin and cytarabine; doxorubicin; checkpoint inhibitors; CAR-T cells; indoleamine 2,3-dioxygenase, hypomethylating agents, 5 azacytidine, decitiabine, cedazuridine, decitiabine combined with cedazuridine (Inqovi, aka Astex727) antiarthritic agents, anti-TNF antibodies.

22. The method of claim 1, wherein the improvement is made by post-treatment patient management selected from a group consisting of: use with therapies associated with pain management; nutritional support; anti-emetics; anti-nausea therapies; anti-anemia therapy; anti-inflammatoire antipyretics; immune stimulants; immune modulants.

23. The method of claim 1, wherein the improvement is made by alternative medicine/therapeutic support selected from one or more of the group consisting of: hypnosis; acupuncture; meditation; herbal medications created either synthetically or through extraction including NF-kappaB inhibitors (such as parthenolide, curcumin, rosmarinic acid); natural anti-inflammatoires (including rhein, parthenolide); immunostimulants (such as those found in Echinacea); antimicrobials (such as berberine); flavonoids and flavones (such as apigenin, genistein); applied kinesiology; energy medicine; photobiomodulation.

24. The method of claim 1, wherein the improvement is made by bulk drug product improvements are selected from a group consisting of: salt formation; homogeneic crystalline structure; pure isomers; increased purity; lower residual solvents and heavy metals.

25. The method of claim 1, wherein the improvement is made by diluents selected from a group consisting of: emulsions; DMSO; NMF; DMF; DMA; ethanol; benzyl alcohol; dextrose containing water for injection; Cremophor; cyclodextrins; PEG.

26. The method of claim 1, wherein the improvement is made by solvent systems selected from a group consisting of: emulsions; DMSO; NMF; DMF; DMA; ethanol; benzyl alcohol; dextrose containing water for injection; Cremophor; PEG.

27. The method of claim 1, wherein the improvement is made by excipients selected from a group consisting of: mannitol; albumin; EDTA; sodium bisulfite; benzyl alcohol; carbonate buffers; phosphate buffers.

28. The method of claim 1, wherein the improvement is made by dosage forms selected from a group consisting of: tablets; capsules; topical gels; topical creams; patches; suppositories; lyophilized dosage fills.

29. The method of claim 1, wherein the improvement is made by dosage kits and packaging selected from a group consisting of: amber vials to protect from light; stoppers with specialized coatings to improve shelf-life stability.

30. The method of claim 1, wherein the improvement is made by drug delivery systems selected from a group consisting of: nanocrystals; bioerodible polymers; liposomes; slow release injectable gels; microspheres.

31. The method of claim 1, wherein the improvement is made by drug conjugate forms selected from a group selected from: polymer systems such as polyethylene glycols; polylactides; polyglycolides; amino acids; peptides; multivalent linkers.

32. The method of claim 1, wherein the improvement is made by compound analogs selected from a group consisting of: alteration of side chains to increase or decrease lipophilicity; additional chemical functionalities to alter reactivity; electron affinity; binding capacity; salt forms; alteration of side chains to increase solubility.

33. The method of claim 1, wherein the improvement is made by prodrug systems selected from a group consisting of: enzyme sensitive esters; dimers; Schiff bases; pyridoxal complexes; caffeine complexes.

34. The method of claim 1, wherein the improvement is made by multiple drug systems selected from a group consisting of: inhibitors of multi-drug resistance; specific drug resistance inhibitors; specific inhibitors of selective enzymes; signal transduction inhibitors; tyrosine kinase inhibitors; DNA repair inhibition; topoisomerase inhibitors with non-overlapping side effects; biological agents with inhibitory effects.

35. The method of claim 1, wherein the improvement is made by biotherapeutic enhancement selected from a group consisting of: biological response modifiers; cytokines; lymphokines; therapeutic antibodies; antisense therapies such as Avastin, Herceptin, Rituxan, and Erbitux; gene therapies; ribozymes; RNA interference; cell based therapeutics.

36. The method of claim 1, wherein the improvement is made by biotherapeutic resistance modulation selected from a group consisting of: biological response modifiers; cytokines; lymphokines; therapeutic antibodies; antisense therapies such as Avastin, Rituxan, Herceptin, Erbitux; gene therapies; ribozymes; RNA interference; cell based therapeutics.

37. The method of claim 1, wherein the improvement is made by radiation therapy enhancement selected from a group consisting of: hypoxic cell sensitizers; radiation sensitizers/protectors; photosensitizers; radiation repair inhibitors; thiol depletion; vaso-targeted agents; use with radioactive seeds, radionuclides, radiolabeled antibodies, brachytherapy.

38. The method of claim 1, wherein the improvement is made by novel mechanisms of action selected from a group consisting of: inhibitors of poly-ADP ribose polymerase; agents that effect vasculature; vasodilation; oncogenic targeted agents; signal transduction inhibitors; tyrosine kinase inhibitors such as imatinib dasatinib, nilotinib, bosutinib; EGFR inhibition; Protein Kinase C inhibition; Phospholipase C down regulation; jun down regulation; histone genes; VEGF; ornithine decarboxylase; jun D; v-jun; GPCR's; protein kinase A; telomerase; prostate specific genes; protein kinases; histone deacetylase; beta-catenin inhibitors; calmodulin antagonists; CaMKII-gamma inhibitors.

39. The method of claim 1, wherein the improvement is made by selective target cell population therapeutics selected from a group consisting of: radiation sensitive cells; radiation resistant cells; energy depleted cells; endothelial cells; cancer stem cells; immunotherapy resistant cells; cells overexpressing beta-catenin; cells overexpressing CaMKII-gamma.

40. A composition administered according to the methods of claim 1, to improve the efficacy and/or reduce the side-effects of suboptimally administered cancer chemotherapy agents comprising administering bisbenzylisoquinolines such as 6,6′,7, 12-tetramethoxy-2,2′-dimethyl-berbaman (tetrandrine) and derivatives in combination with other therapeutic agents selected from a group consisting of: signal transduction inhibitors and tyrosine kinase inhibitors such as imatinib, dasatinib, nilotinib and bosutinib; in combination with one or more of topoisomerase inhibitors; use with fraudulent nucleosides; use with fraudulent nucleotides; use with thymidylate synthase inhibitors; use with cisplatin or platinum analogs; use with amino acid substituted alkylating agents such as the nitrosoureas (BCNU, Gliadel wafers, CCNU), bendamustine (Treanda); use with anti-tubulin agents; use with antimetabolites; use with berberine; apigenin; amonafide; colchicine and analogs; genistein; etoposide; cytarabine; campothecins; vinca alkaloids; topoisomerase inhibitors; 5-fluorouracil; gemcitabine; curcumin; NFKB inhibitors; rosmarinic acid; In combination with biological therapies such as antibodies such as Avastin, Rituxan, Herceptin, Erbitux; PD-1 and PDL-1 inhibitors; prendimustine; DNA and RNA therapeutics; Braf inhibitors; BTK inhibitors; 5-azacytidine; decitabine; indoleamine 2,3-dioxygenase.

41. The composition of claim 40, wherein the improvement is made by administration of tetrandrine and/or derivatives and without other therapeutic agents; and by administration of an agent affecting at least one factor and/or at least one parameter contributing to the improvement by also administering at least one agent imparting one or more properties selected from the group consisting of: chemosensitization, chemopotentiation, bulk drug product improvement(s), diluent(s), solvent system(s), excipient(s), dosage form(s), dosage kit(s), drug delivery, drug conjugate(s), compound analog(s) and prodrug system(s).
